(19) United States
(12) Patent Application Publication
(10) Pub. No.: US 2005/0228023 A1
(43) Pub. Date: Oct. 13, 2005

(54) Agonist and antagonist ligands of the nociceptin receptor

(75) Inventors: Nurulain T. Zaveri, Saratoga, CA (US); Faming Jiang, Mountain View, CA (US); Cris M. Olsen, Soquel, CA (US); Willma Polgar, Sunnyvale, CA (US); Lawrence Toll, Redwood City, CA (US)

Correspondence Address: REED INTELLECTUAL PROPERTY LAW GROUP, 1400 PAGE MILL ROAD, PALO ALTO, CA, 94304-1124, US

(73) Assignee: SRI International, Menlo Park, CA (US)

(21) Appl. No.: 11/019,738

(22) Filed: Dec. 20, 2004

Related U.S. Application Data
(60) Provisional application No. 60/530,969, filed on Dec. 19, 2003.

(51) Int. Cl. A61K 31/454; A61K 31/404

(52) U.S. Cl. 514/323; 514/418

(57) ABSTRACT

Novel piperidyl indolinone compounds are provided that are useful as agonist and antagonist ligands of the nociceptin receptor. The compounds are useful to treat a variety of disorders, and are particularly useful as analgesics

### CROSS-REFERENCE TO RELATED APPLICATION

This application claims priority under 35 U.S.C. §1 19(e)(1) to provisional U.S. Patent Application Serial No. 60/530,969, filed Dec. 19, 2003, the disclosure of which is incorporated by reference herein.

### ACKNOWLEDGEMENT OF GOVERNMENT SUPPORT

This invention was made with United States government support under Grant number DA14026 awarded by the National Institute on Drug Abuse. Accordingly, the United States government has certain rights to this invention.

### TECHNICAL FIELD

This invention relates to indolinone derivatives that are useful as agonist and antagonist ligands of the nociceptin receptor and thus have analgesic properties.

### BACKGROUND

The discovery of a fourth member of the opioid receptor family, opioid receptor-like (ORL 1) receptor (now also called the nociceptin receptor) has been significant, in large part because although this new G-protein coupled receptor was clearly in the opioid family, it did not bind opiates with high affinity. The endogenous ligand for this receptor is a 17-amino acid neuropeptide named nociceptin (NC) by Meunier et al. (1995) Nature 377:532-535, and named orphanin FQ (N/OFQ) by Reinscheid et al. (1995) Science 270:792-794.

The ORL1 receptor and its N/OFQ ligand are widely distributed in the brain and central nervous system (CNS), as well as in the periphery. The N/OFQ-ORL1 system is believed to have a role in pain, particularly in the modulation of analgesia and tolerance to opiate analgesics, anxiety, learning, memory, food intake, diuresis, and drug addiction. In particular, N/OFQ has been shown to inhibit the release of several neurotransmitters, including serotonin and dopamine, implicating this peptide in the inhibition of reward pathways in drug addiction and thus points to a potential utility of ORL1 agonists as anxiolytics and opiate/drug abuse treatments. There have been several studies which clearly demonstrate that central administration of N/OFQ abolishes ethanol-induced conditioned place preference and can prevent stress-induced ethanol-seeking behavior in rats (Ciccocioppo et al. (1999) Psychopharmacology 141:220-224; Martin-Fardon et al. (2000) Neuroreport 11:1939-1943). Furthermore, recent reports have shown that N/OFQ is able to abolish morphine-induced conditioned place preference (Ciccocioppo et al. (2000) Eur. J. Pharmacol. 404:153-159) and inhibit morphine withdrawal symptoms induced by naloxone (Kotlinska et al. (2000) Life Sci. 66:119-123). Studies also suggest a role for ORL1 agonists in asthma and cough treatment and for ORL1 antagonists as nootropics, anorectics, and analgesics. Studies also suggest a role for ORL1 agonists and antagonists outside of the central nervous system, for example for selective renal diuretic and antinatriuretic effects (U.S. Pat. No. 5,840,696 to Kapusta et al.; U.S. Patent Publication No. 20030040472 to Larsen et al.; Kapusta et al. (2002) FASEB Journal 16:A841).

From the myriad of modulatory actions of N/OFQ in several neurological pathways, it is clear that ORL1 represents an important new molecular target for the development of novel therapeutics, and much research has been conducted in developing both agonist and antagonist non-peptide ligands for the ORL1 receptor as potential drugs for various human disorders. A review article on the subject, Zaveri (2003) Life Sciences 73:663-678, describes these in detail.

Numerous peptide ligands have been developed, based upon the N/OFQ heptadecapeptide. These are described, for example, in Dooley et al (1997) Journal of Pharmacology and Experimental Therapeutics 283:735-741; Calo et al. (1998) Journal of Medicinal Chemistry 41:3360-3366; Guerrini et al. (2000) Journal of Medicinal Chemistry 43:2805-2813; Okada et al. (2000) Biochemical and Biophysical Research Communications 278:493-498; Guerrini et al. (2001) Journal of Medicinal Chemistry 44:3956-3964; Ambo et al. (2001) Journal of Medicinal Chemistry 44:4015-4018; Becker et al. (1999) Journal of Biological Chemistry 274:27513-27522; Calo et al. (2002) British Journal of Pharmacology 136:303-311; and WO 01/98324 to Larsen et al. Although peptide ligand research has helped to understand the N/OFQ-ORL1 system, however, it has become apparent that the therapeutic utility of ORL1 ligands, particularly for neurological disorders, can only be realized with potent non-peptide ligands, which are more likely to penetrate the CNS than peptides and can be more easily developed as drugs.

Recent research has, accordingly, been directed towards developing potent non-peptide agonists and antagonists. Since the ORL1 receptor belongs to the opioid class of receptors, small-molecule opiate ligand have been examined for binding at ORL1: the σ receptor ligands carbetapentane and rimcazole (Kobayashi et al. (1997) British Journal of Pharmacology 120:986-987); the μ-selective opiates lofentanil, an anilidopiperidine, and etorphine, an oripavine derivative (Butour et al. (1997) European Journal of Pharmacology 321:97-103); anilidopiperidines, morphinans, and benzomorphan classes of opiate ligands (Hawkinson et al. (2000) European Journal of Pharmacology 389:107-114); the 5-HT partial agonist spiroxatrine, the neuroleptic pimozide, and the partial μ agonist buprenorphine (Zaveri et al. (2001) European Journal of Pharmacology 428:29-36). Another opiate that has served as a lead for the design of selective ORL1 ligands is the morphinan naloxonebenzoylhydrazone, which is a κ opioid agonist and μ antagonist and has an antinociceptive effect in vivo (Gistrak et al. (1989) Journal of Pharmacology and Experimental Therapeutics 251:469-476).

The nonselective opiate ligands have thus far provided useful leads for the design of selective ORL1 ligands. These nonpeptide ligands, both agonists and antagonists, can be broadly divided into the following five structural classes: morphinan-based ligands; benzimidazopiperidines; spiropiperidines; aryl piperidines; and 4-aminoquinolines.

Morphinan-based ligands: U.S. Pat. No. 5,834,478 to Ito describes 6-substituted morphinan hydroxamic acids having ORL1 antagonist activity and agonist activity at the μ, δ, and κ opioid receptors. Seki et al. (1999) European Journal of Pharmacology 376:159-167 describe a morphinan κ agonist, a 6-N-methylamido morphinan, which is structurally very similar to the Ito hydroxamic acids.

Benzimidazopiperidines: WO 98/54168 to Ozaki et al. describes the first nonpeptide pure ORL1 antagonists, which are benzimidazolinones of the general formula 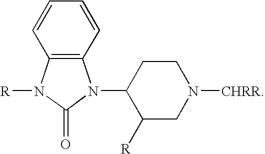
Benzimidazolinones are also described in International Patent Publication WO 99/36421 to Ito et al., International Patent Publication WO 01/39775 to Kyle et al, U.S. Pat. No. 6,172,067 to Ito et al., EP 1122257 to Ito et al., and U.S. Pat. No. 6,340,681 to Ito. In the compounds described in these references.

Spiropiperidines: EP 0856514 to Adam et al. describes a series of 1,3,8-triazaspiro[4,5]decan-4-ones having the general structure

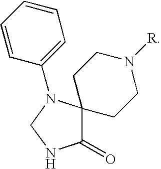

Compounds containing an alicyclic substituent such as cyclododecyl or 4-isopropylcyclohexyl at the piperidine nitrogen have also been reported as agonists, in Rover et al. (2000) Journal of Medicinal Chemistry 43:1329-1338. Additional agonists are described in U.S. Pat. No. 6,075,034 to Adam et al. (spiropiperidines modified within the imidazole ring) and U.S. Pat. No. 6,113,527 to Adam et al. (diazaspiro[3,5]nonane derivatives). Further modification of the heterocyclic portion as well as the piperidine N-substituent has resulted in a series of spirofused benzofuranone antagonists, as described in U.S. Pat. No. 6,166,209 to Adam et al. U.S. Pat. No. 6,277,991 to Hohlweg et al. describes the synthesis and characterization of 1,3,8-triazaspirodecanones as ORL ligands. EP 0997464 to Ito et al., JP 2000169476 to Kawamoto et al., and International Patent Publications WO 00/34280 and WO 01/96337, both to Satoh et al., also describe triazaspirodecanones as ORL agonists and antagonists.

Aryl piperidines: While the earlier reported ligands were substituted with spiro- and benzo-fused heterocycles at the 4-position of the central piperidine ring, U.S. Pat. No. 6,262,066 to Tulshian et al. describes a series of phenylpiperidines as ORL1 agonists, which also encompass the benzimidazolinones and triazaspirodecanones. WO 00/14067 to Cesura et al. and WO 00/27815 to Barlocco et al. also describe phenylpiperidines as ORL1 ligands.

4-Aminoquinolines: This class of ORL ligands is described in International Patent Publication No. WO 99/48492 to Shinkai et al.

However, despite the advances in the art, there remains is a continuing need to identify small-molecule ORL1 ligands to aid in the development of potent non-peptide agonists and antagonists. The present invention addresses those needs by the development of indolinone derivatives, such as piperidyl indolinones with modified piperidine N-substituents, that provide both potent agonists as well as antagonists.

### SUMMARY OF THE INVENTION

One aspect of the invention relates to a method for modulating a process mediated by the nociceptin receptor, comprising administering to a mammalian subject an amount of an indolinone derivative effective to modulate the process, wherein the indolinone derivative is an indolin-2-one compound N-substituted with a nitrogen-containing alicyclic group, wherein the alicyclic nitrogen atom is optionally substituted with a mono-, bi- or tri-cyclic hydrocarbyl group.

Another aspect of the invention pertains to a method for modulating the nociceptin receptor in a patient in need of such modulation, comprising administering to the patient a therapeutically effective amount of a compound of formula (I)

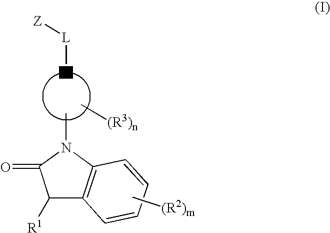

or a pharmacologically active equivalent thereof, wherein:

m is an integer in the range of zero to 3 inclusive;

n is an integer in the range of zero to 2 inclusive; 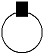
is a nitrogen-containing heterocyclic group having 3-14 carbon atoms;

R¹ is selected from hydrogen, hydroxyl, halo, haloalkyl, amino, aminoalkyl, aminocarbonyl, alkylamino, dialkylamino, alkyl, alkenyl, alkoxy, alkoxycarbonyl, aryl, and aralkyl, or can be taken together with R² to form a cyclic group;

R² is selected from hydroxyl, halo, haloalkyl, amino, aminoalkyl, aminocarbonyl, alkylamino, dialkylamino, alkyl, alkenyl, alkoxy, alkoxycarbonyl, alkylthio, aryloxy, aryl, aralkyl, arylthio, carboxy, cycloalkyl, cycloalkylalkyl, heteroaryl, and heteroarylalkyl, or two R² substituents taken together can form a cyclic structure, and further wherein when m is greater than 1, the R² groups may be the same or different;

R³ is selected from hydrogen, hydroxyl, hydroxyalkyl, halo, haloalkyl, alkylidene, amino, aminoalkyl, aminocarbonyl, alkylamino, dialkylamino, alkyl, alkenyl, alkynyl, alkoxy, carboxy, carboxyalkyl, alkoxycarbonyl, alkoxycarbonylalkyl, carbamoyl, carbamoylalkyl, alkylcarbamoyl, alkylcarbamoylalkyl, dialkylcarbamoyl, dialkylcarbamoylalkyl, carbamoylaminoalkyl, alkylcarbamoylaminoalkyl, dialkylcarbamoylaminoalkyl, carbamoyloxyalkyl, alkylcarbamoyloxyalkyl, dialkylcarbamoyloxyalkyl, alkylsulfonylaminoalkyl, aminosulfonylaminoalkyl, alkylaminosulfonylaminoalkyl, dialkylaminosulfonylaminoalkyl, and heteroaryl, and wherein when n is 2, the two R³ groups may be the same or different;

L is —(CHR⁴)ₚ—, —CH₂—Xᵥ—, or —C(═NH)—NR⁵—;

p is an integer in the range of zero to 4 inclusive;

v is an integer in the range of 1 to 3 inclusive;

R⁴ is selected from hydrogen, hydroxyl, halo, amino, aminoalkyl, alkylamino, dialkylamino, alkyl, cycloalkyl, cycloalkylalkyl, alkenyl, alkoxy, aryl and aralkyl, wherein when p is greater than 1, the R⁴ may be the same or different;

R⁵ is selected from hydrogen, alkyl, cycloalkyl, cycloalkylalkyl, alkenyl, alkoxy, aryl and aralkyl;

X is independently CH₂, NR⁶ or O, wherein at least one X, if v is greater than 1, is NR⁶ or O;

R⁶ is selected from hydrogen, alkyl, cycloalkyl, cycloalkylalkyl, alkenyl, alkoxy, aryl and aralkyl, and. In some preferred embodiments, when L is —(CHR⁴)ₚ—, p is 0 and so the R⁴ group; and

Z is selected from hydrogen and cyclic hydrocarbyl, optionally substituted with one or more substituents, with the proviso that when p is zero, , or a mono-, bi- or tri-cyclic hydrocarbyl, optionally substituted with one or more substituents, with the proviso that when p is zero, Z is other than hydrogen.

Yet another aspect of the invention relates to the use of the N-substituted indolin-2-one compounds of the invention in methods for treating an individual with a drug or alcohol abuse disorder, for treating an individual suffering from pain, and for preventing or treating anxiety and stress disorders in an individual, where the method comprises administering a therapeutically effective amount of a compound of the invention to the individual.

Still another aspect of the invention relates to the use of the N-substituted indolin-2-one compounds of the invention in methods for treating or preventing eating disorders, schizophrenia, Parkinsonism, depression, cognitive dysfunction, and in a method for the amelioration of reduced cognitive brain function, where the method comprises administering a therapeutically effective amount of a compound of the invention to the individual.

Yet another aspect of the invention relates to the use of the N-substituted indolin-2-one compounds of the invention in methods for treating or preventing hyponatremia, hypokalemia, a water retaining condition, multiple organ failure, hypertension, and edema, where the method comprises administering a therapeutically effective amount of a compound of the invention to the individual.

Another aspect of the invention pertains to an indolin-2-one compound N-substituted with a nitrogen-containing alicyclic group, wherein the alicyclic nitrogen atom is optionally substituted with a mono-, bi- or tri-cyclic hydrocarbyl group.

A further aspect of the invention relates to a pharmaceutical composition comprising a therapeutically effective amount of a compound of the invention and a pharmaceutically acceptable carrier.

### DETAILED DESCRIPTION OF THE INVENTION

I. Definitions and Nomenclature

The present invention relates to a series of indolinone derivatives, preferably piperidyl indolinones substituted on the nitrogen atom of the piperidinyl ring. These compounds are potent agonists or antagonists of the nociceptin receptor. Antagonist or agonist activity is determined by the degree of inhibition or the degree of stimulation, respectively, of the nociceptin receptor.

It must be noted that, as used in the specification and the appended claims, the singular forms “a,” “an,” and “the” include plural referents unless the context clearly dictates otherwise. Thus, for example, reference to “a compound” (e.g., as in a pharmaceutical composition of the invention) includes not only a single compound but combinations of compounds, reference to “a pharmaceutical carrier” includes combinations or mixtures of carriers as well as a single carrier, and the like.

In this specification and in the claims which follow, reference will be made to a number of terms which shall be defined to have the following meanings.

The term “alkaryl” refers to an aryl group with an alkyl substituent, and the term “aralkyl” refers to an alkyl group with an aryl substituent, wherein “aryl” and “alkyl” are as defined above. Preferred aralkyl groups contain 6 to 24 carbon atoms, and particularly preferred aralkyl groups contain 6 to 16 carbon atoms. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. Alkaryl groups include, for example, p-methylphenyl, 2,4-dimethylphenyl, p-cyclohexylphenyl, 2,7-dimethylnaphthyl, 7-cyclooctylnaphthyl, 3-ethyl-cyclopenta-1,4-diene, and the like.

The term “alkenyl” as used herein refers to a linear, branched or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein contain 2 to about 18 carbon atoms, preferably 2 to 12 carbon atoms. The term “lower alkenyl” intends an alkenyl group of 2 to 6 carbon atoms, and the specific term “cycloalkenyl” intends a cyclic alkenyl group, preferably having 5 to 8 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkoxy” as used herein intends an alkyl group bound through a single, terminal ether linkage; that is, an “alkoxy” group may be represented as —O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Preferred substituents identified as “C₁₋₆ alkoxy” or “lower alkoxy” herein contain 1 to 3 carbon atoms, and particularly preferred such substituents contain 1 or 2 carbon atoms (i.e., methoxy and ethoxy).

The term “alkyl” as used herein refers to a branched or unbranched saturated hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl, and the like. Generally, although again not necessarily, alkyl groups herein contain 1 to about 18 carbon atoms, preferably 1 to about 12 carbon atoms. The term “lower alkyl” intends an alkyl group of 1 to 6 carbon atoms. Preferred substituents identified as “C₁₋₆ alkyl” or “lower alkyl” contain 1 to 3 carbon atoms, and particularly preferred such substituents contain 1 or 2 carbon atoms (i.e., methyl and ethyl). “Substituted alkyl” refers to alkyl substituted with one or more substituent groups, and the terms “heteroatom-containing alkyl” and “heteroalkyl” refer to alkyl in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “alkylidene” refers to a linear or branched alkylidene group containing 1 to about 6 carbon atoms, such as methylene, ethylidene, propylidene, isopropylidene, butylidene, and the like.

The term “alkynyl” as used herein refers to a linear or branched hydrocarbon group of 2 to 24 carbon atoms containing at least one triple bond, such as ethynyl, n-propynyl, and the like. Generally, although again not necessarily, alkynyl groups herein contain 2 to about 18 carbon atoms, preferably 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 20 carbon atoms, and particularly preferred aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. If not otherwise indicated, the term “aryl” includes unsubstituted, substituted, and/or heteroatom-containing aromatic substituents.

The term “aryloxy” as used herein refers to an aryl group bound through a single, terminal ether linkage, wherein “aryl” is as defined above. An “aryloxy” group may be represented as —O-aryl where aryl is as defined above. Preferred aryloxy groups contain 5 to 20 carbon atoms, and particularly preferred aryloxy groups contain 5 to 14 carbon atoms. Examples of aryloxy groups include, without limitation, phenoxy, o-halo-phenoxy, m-halo-phenoxy, p-halo-phenoxy, o-methoxy-phenoxy, m-methoxy-phenoxy, p-methoxy-phenoxy, 2,4-dimethoxy-phenoxy, 3,4,5-trimethoxy-phenoxy, and the like.

The term “cyclic” refers to an alicyclic or aromatic substituent, group, or compound that may or may not be substituted and/or heteroatom containing, and that may be monocyclic, bicyclic, or polycyclic.

The terms “halo” is used in the conventional sense to refer to a chloro, bromo, fluoro or iodo substituent.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

“Hydrocarbyl” refers to univalent hydrocarbyl radicals containing 1 to about 30 carbon atoms, preferably 1 to about 24 carbon atoms, more preferably 1 to about 18 carbon atoms, most preferably about 1 to 12 carbon atoms, including linear, branched, cyclic, saturated, and unsaturated species, such as alkyl groups, alkenyl groups, aryl groups, and the like. “Substituted hydrocarbyl” refers to hydrocarbyl substituted with one or more substituent groups, and the term “heteroatom-containing hydrocarbyl” refers to hydrocarbyl in which at least one carbon atom is replaced with a heteroatom. Unless otherwise indicated, the term “hydrocarbyl” is to be interpreted as including unsubstituted, substituted, non-heteroatom-containing and heteroatom-containing hydrocarbyl moieties.

The term “saturated” is intended to include both fully saturated compounds, in particular ring structures such as cycloalkyl groups, as well as partially saturated compounds such as cycloalkenyl groups. For example, tetrahydronaphthyl is a partially saturated ring system and therefore is considered a “saturated” ring system herein. The term “unsaturated” refers to fully unsaturated moieties.

By “substituted” as in “substituted hydrocarbyl” and the like, as alluded to in some of the aforementioned definitions, is meant that in the hydrocarbyl or other moiety, at least one hydrogen atom bound to a carbon (or other) atom is replaced with one or more non-hydrogen substituents. Examples of such substituents include, without limitation: functional groups such as halo, hydroxyl, sulfhydryl, C₁₋₂₄ alkoxy, C₂₋₂₄ alkenyloxy, C₂₋₂₄ alkynyloxy, C₅₋₂₀ aryloxy, acyl (including C₂₋₂₄ alkylcarbonyl (—CO-alkyl) and C₆₋₂₀ arylcarbonyl (—CO-aryl)), acyloxy (—O-acyl), C₂₋₂₄ alkoxycarbonyl (—(CO)—O-alkyl), C₆₋₂₀ aryloxycarbonyl (—(CO)—O-aryl), halocarbonyl (—CO)—X where X is halo), C₂₋₂₄ alkylcarbonato (—O—(CO)—O-alkyl), C₆₋₂₀ arylcarbonato (—O—(CO)—O-aryl), carboxy (—COOH), carboxylate (—COO⁻), carbamoyl (—(CO)—NH₂), mono-(C₁₋₂₄ alkyl)-substituted carbamoyl (—(CO)—NH(C₁₋₂₄ alkyl)), di-(C₁₋₂₄ alkyl)-substituted carbamoyl (—(CO)—N(C₁₋₂₄ alkyl)₂), mono-substituted arylcarbamoyl (—(CO)—NH-aryl), thiocarbamoyl (—(CS)—NH₂), carbamido (—NH—(CO)—NH₂), cyano(—C≡N), isocyano (—N⁺≡C⁻), cyanato (—O—C≡N), isocyanato (—O—N⁺≡C⁻), isothiocyanato (—S—C≡N), azido (—N═N⁺═N⁻), formyl (—(CO)—H), thioformyl (—(CS)—H), amino (—NH₂), mono- and di-(C₁₋₂₄ alkyl)-substituted amino, mono- and di-(C₅₋₂₀ aryl)-substituted amino, C₂₋₂₄ alkylamido (—NH—(CO)-alkyl), C₆₋₂₀ arylamido (—NH—(CO)-aryl), imino (—CR═NH where R is hydrogen, C₁₋₂₄ alkyl, C₅₋₂₀ aryl, C₆₋₂₄ alkaryl, C₆₋₂₄ aralkyl, etc.), alkylimino (—R═N(alkyl), where R=hydrogen, alkyl, aryl, alkaryl, etc.), arylimino (—CR═N(aryl), where R is hydrogen, alkyl, aryl, alkaryl, etc.), nitro (—NO₂), nitroso (—NO), sulfo (—SO₂—OH), sulfonato (—SO₂—O⁻), C₁₋₂₄ alkylsulfanyl (—S-alkyl; also termed “alkylthio”), arylsulfanyl (—S-aryl; also termed “arylthio”), C₁₋₂₄ alkylsulfinyl (—(SO)-alkyl), C₅₋₂₀ arylsulfinyl (—(SO)-aryl), C₁₋₂₄ alkylsulfonyl (—SO₂-alkyl), C₅₋₂₀ arylsulfonyl (—SO₂-aryl), phosphono (—P(O)(OH)₂), phosphonato (—P(O)(O⁻)₂), phosphinato (—P(O)(O⁻)), phospho (—PO₂), and phosphino (—PH₂); and the hydrocarbyl moieties C₁₋₂₄ alkyl (preferably C₁₋₁₈ alkyl, more preferably C₁₋₁₂ alkyl, most preferably C₁₋₆-alkyl), C₂₋₂₄ alkenyl (preferably C₂₋₁₈ alkenyl, more preferably C₂₋₁₂ alkenyl, most preferably C₂₋₆ alkenyl), C₂₋₂₄ alkynyl (preferably C₂₋₁₈ alkynyl, more preferably C₂₋₁₂ alkynyl, most preferably C₂₋₆ alkynyl), C₅₋₂₀ aryl (preferably C₅₋₁₄ aryl), C₆₋₂₄ alkaryl (preferably C₆₋₁₈ alkaryl), and C₆₋₂₄ aralkyl (preferably C₆₋₁₈ aralkyl).

In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

When the term “substituted” appears prior to a list of possible substituted groups, it is intended that the term apply to every member of that group. For example, the phrase “substituted alkyl, alkenyl, and aryl” is to be interpreted as “substituted alkyl, substituted alkenyl, and substituted aryl.” Analogously, when the term “heteroatom-containing” appears prior to a list of possible heteroatom-containing groups, it is intended that the term apply to every member of that group. For example, the phrase “heteroatom-containing alkyl, alkenyl, and aryl” is to be interpreted as “heteroatom-containing alkyl, substituted alkenyl, and substituted aryl.”

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present. Similarly, the phrase an “optionally present” double bond as indicated by a dotted line - - - in the chemical formulae herein means that a double bond may or may not be present, and, if absent, a single bond is indicated.

When referring to a compound of the invention, applicants intend the term “compound” to encompass riot only the specified molecular entity but also its pharmaceutically acceptable, pharmacologically active equivalents, including, but not limited to, salts, esters, amides, prodrugs, conjugates, active metabolites, and other such derivatives, analogs, and related compounds.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. For example, treatment of a patient with a compound of the invention can involve treatment of an individual who exhibits symptoms of a particular adverse condition or disorder as well as prevention of the adverse condition or disorder in an asymptomatic individual susceptible to developing the adverse condition or disorder, e.g., as a result of genetic predisposition, environmental factors, or the like.

By the terms “effective amount” and “therapeutically effective amount” of a compound of the invention is meant a nontoxic but sufficient amount of the drug or agent to provide the desired effect. In particular, this is intended to include an amount effective to modulate the nociceptin receptor, a nociceptin receptor antagonistic effective amount and a nociceptin receptor agonistic effective amount.

By “pharmaceutically acceptable” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration. “Pharmacologically active” (or simply “active”) as in a “pharmacologically active” derivative or analog, refers to a derivative or analog having the same type of pharmacological activity as the parent compound and approximately equivalent in degree.

II. The Compounds of the Invention

The compounds of the invention are indolin-2-one derivatives that are N-substituted with a nitrogen-containing alicyclic group, wherein the nitrogen atom of the alicyclic group is optionally substituted with a mono-, bi- or tri-cyclic hydrocarbyl group.

In general, the indolinone derivatives of the invention have the structure of formula (I) or are pharmacologically active equivalents thereof

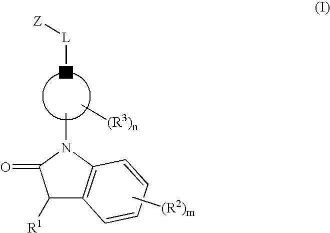

In formula (I):

m is an integer in the range of zero to 3 inclusive;

n is an integer in the range of zero to 2 inclusive; 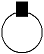
is a nitrogen-containing heterocyclic group having 3-14 carbon atoms. Piperidyl is one preferred such group.

R¹ is selected from hydrogen, hydroxyl, halo, haloalkyl, amino, aminoalkyl, aminocarbonyl, alkylamino, dialkylamino, alkyl, alkenyl, alkoxy, alkoxycarbonyl, aryl, and aralkyl, or can be taken together with R² to form a cyclic group. Preferred R¹ substituents include hydrogen and alkyl.

R² is selected from hydroxyl, halo, haloalkyl, amino, aminoalkyl, aminocarbonyl, alkylamino, dialkylamino, alkyl, alkenyl, alkoxy, alkoxycarbonyl, alkylthio, aryloxy, aryl, aralkyl, arylthio, carboxy, cycloalkyl, cycloalkylalkyl, heteroaryl, and heteroarylalkyl, or two R² substituents taken together can form a cyclic structure. When m is greater than 1, the R² groups may be the same or different. In some preferred embodiments, m is zero and the R² groups are absent. However, when m is 1, 2, or 3, preferred R² groups include alkyl and alkyloxycarbonyl.

R³ is selected from hydrogen, hydroxyl, hydroxyalkyl, halo, haloalkyl, alkylidene, amino, aminoalkyl, aminocarbonyl, alkylamino, dialkylamino, alkyl, alkenyl, alkynyl, alkoxy, carboxy, carboxyalkyl, alkoxycarbonyl, alkoxycarbonylalkyl, carbamoyl, carbamoylalkyl, alkylcarbamoyl, alkylcarbamoylalkyl, dialkylcarbamoyl, dialkylcarbamoylalkyl, carbamoylaminoalkyl, alkylcarbamoylaminoalkyl, dialkylcarbamoylaminoalkyl, carbamoyloxyalkyl, alkylcarbamoyloxyalkyl, dialkylcarbamoyloxyalkyl, alkylsulfonylaminoalkyl, aminosulfonylaminoalkyl, alkylaminosulfonylaminoalkyl, dialkylaminosulfonylaminoalkyl, and heteroaryl, and wherein when n is 2, the two R³ groups may be the same or different. In some preferred embodiments, n is zero and the R³ groups are thus absent. However, when n is 1 or 2, preferred R³ groups include alkyl and alkyloxycarbonyl.

L is —(CHR⁴)ₚ—, —CH₂—Xᵥ—, or —C(═NH)—NR⁵— where p is an integer in the range of zero to 4 inclusive, R⁴ is selected from hydrogen, hydroxyl, halo, amino, aminoalkyl, alkylamino, dialkylamino, alkyl, cycloalkyl, cycloalkylalkyl, alkenyl, alkoxy, aryl and aralkyl, wherein when p is greater than 1, the R⁴ may be the same or different, v is an integer in the range of 1 to 3 inclusive, R⁵ is selected from hydrogen, alkyl, cycloalkyl, cycloalkylalkyl, alkenyl, alkoxy, aryl and aralkyl, X is independently CH₂, NR⁶ or O, wherein at least one X, if v is greater than 1, is NR⁶ or O, and R⁶ is selected from hydrogen, alkyl, cycloalkyl, cycloalkylalkyl, alkenyl, alkoxy, aryl and aralkyl. In some preferred embodiments, when L is —(CHR⁴)ₚ—, p is zero and the R⁴ group is absent. However, when p is 1, 2, 3, or 4, preferred R⁴ groups include hydrogen and alkyl, in particular C₁₋₆ alkyl such as methyl and ethyl, and cycloalkyl such as cyclopropyl and cyclohexyl. Examples of the —CH₂—Xᵥ— linker include —CH₂—O—, —CH₂—O—CH₂—, —CH₂—CH₂—O—, —CH₂—NH—CH₂—, —CH₂—CH₂—N(CH₃)—CH₂—, and so forth.

Z is hydrogen or cyclic hydrocarbyl, including mono-, bi-, and tri-cyclic hydrocarbyl, optionally substituted with one or more substituents, with the proviso that when p is zero, Z is other than hydrogen.

Exemplary such substituents include, without limitation, hydroxyl, halo, haloalkyl, amino, aminoalkyl, alkylamino, dialkylamino, alkyl, alkenyl, alkoxy, aryl, and aralkyl.

Exemplary nitrogen heterocycles include, by way of illustration and not limitation, the following monocyclic moieties: 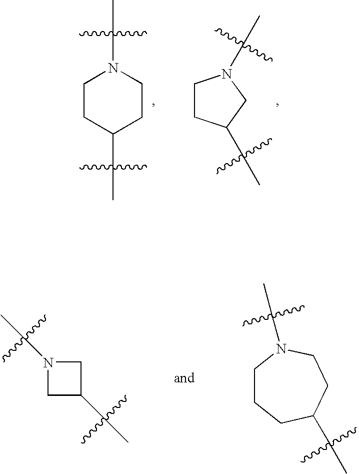
with piperidyl being one preferred embodiment.

Exemplary bicyclic nitrogen heterocycles, by way of illustration and not limitation, 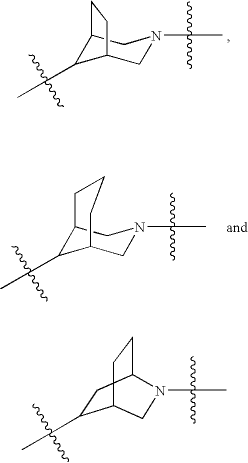
with SR 16492 representing one specific example of such a group;

Exemplary Z groups include, by way of illustration and not limitation, saturated rings such as: 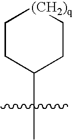
where q is an integer from 0-5. Examples of compounds with saturated mono-cyclic hydrocarbyl groups as the “Z” substituent are compounds SR 14148, SR 16476 and SR 16477, described in Example 3, as well as compounds SR 14150 and SR 16501, described in Example 5. The monocyclic hydrocarbyl group can also be an unsaturated ring such as a phenyl substituent.

One example of a compound with an unsaturated monocyclic hydrocarbyl group as the “Z” substituent, is compound SR 16412 in Example 2. The unsaturated mono-cyclic hydrocarbyl group can also be substituted with a saturated ring as follows: 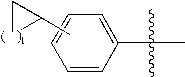
where t is an integer from 1-6. This is exemplified by compound SR 16410 in Example 3 (t=4).

Exemplary bicyclic hydrocarbyl groups suitable as Z include, by way of illustration and not limitation, fused rings having the structure: 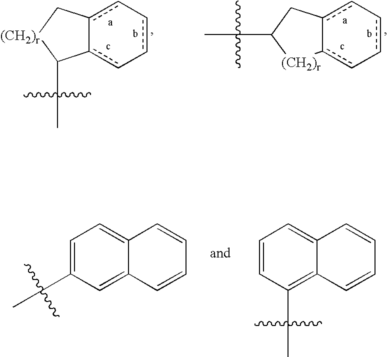
where r is an integer from 1-2, and a, b, and c, are optional double bonds. Examples of these types of bi-cyclic hydrocarbyl groups are present in compound SR 16405 in Example 3 and compound SR 16439 in Example 5, where r is 2 and a, b, and c are double bonds; and in isomer compounds SR 16406 and SR 16407 in Example 3, and compound SR 16408 in Example 5, where r is 2 and a, b, and c are absent, i.e., the ring has all single bonds). Other fused rings that are suitable bi-cyclic hydrocarbyl groups include: 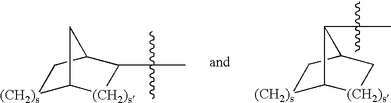
where s and s′ are integers from 0-2. Examples of the former (where s and s′ are 1) include isomer compounds SR 16432 and SR 16433 in Example 3 and compound SR 16490 in Example 5, and (where s is 0 and s' is 2) compound SR 16414 in Example 3. Examples of the latter (where s and s′ are 2) include compound SR 16434 in Example 3.

Exemplary tricyclic hydrocarbyl groups suitable as Z include, by way of illustration and not limitation: 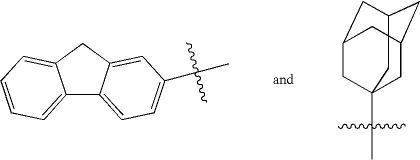
the former being exemplified in compound SR 16411, in Example 5.

More specifically, the indolinone derivatives of the invention can be piperidyl indolinones with modified piperidine N-substituents. In particular, the piperidyl nitrogen is substituted with a mono-, bi- or tri-cyclic hydrocarbyl. These compounds are potent agonists or antagonists of the nociceptin receptor. Antagonist or agonist activity is determined by the degree of inhibition or the degree of stimulation, respectively, of the nociceptin receptor. In one preferred embodiment, the compounds of the invention have a reasonably high affinity for the nociceptin receptor, with a Kᵢ value of less than 1.0 μM. More preferred compounds have a Kᵢ value of less than 50 nanomolar.

In one embodiment of the invention, the N-substituted indolin-2-one compound of the invention is piperidin-4-yl-1,3-dyhydro-indol-2-one, which has the following structure.

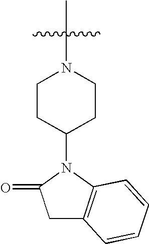

The piperidyl and indolinone rings may also have one or more substituents. Exemplary substituents suitable for the piperidyl ring include, by way of illustration and not limitation, hydrogen, hydroxyl, hydroxyalkyl, halo, alkylidene, amino, aminoalkyl, aminocarbonyl, alkylamino, dialkylamino, alkyl, alkenyl, alkynyl, alkoxy, carboxy, carboxyalkyl, alkoxycarbonyl, alkoxycarbonylalkyl, aryl, aralkyl, carbamoyl, carbamoylalkyl, alkylcarbamoyl, alkylcarbamoylalkyl, dialkylcarbamoyl, dialkylcarbamoylalkyl, carbamoylaminoalkyl, alkylcarbamoylaminoalkyl, dialkylcarbamoylaminoalkyl, carbamoyloxyalkyl, alkylcarbamoyloxyalkyl, dialkylcarbamoyloxyalkyl, alkylsulfonylaminoalkyl, aminosulfonylaminoalkyl, alkylaminosulfonylaminoalkyl, dialkylaminosulfonylaminoalkyl, and an aromatic heterocyclic group. Exemplary substituents suitable for the indolinone ring include, by way of illustration and not limitation, hydroxyl, halo, amino, aminoalkyl, aminocarbonyl, alkylamino, dialkylamino, alkyl, alkenyl, alkoxy, alkyloxycarbonyl, alkylthiol, aryloxy, aryl, aralkyl, arylthio, carboxy, cycloalkyl, cycloalkylalkyl, heteroaryl, and heteroarylalkyl. For non-therapeutic applications, e.g., for screening or assays, the piperidyl and indolinone rings may also be substituted with a haloalkyl group.

Compounds of particular interest include those having formulas (Ia), (lb), (Ic), (Id), and (Ie) as shown below, where the nitrogen-containing alicyclic group, R¹, R², R³, m and n, L, Z, are as defined herein. Compound (Ia) has the structure: 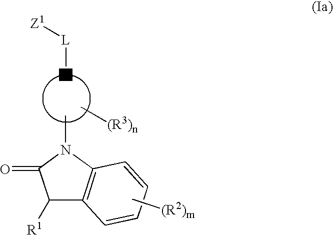
where Z¹ is a mono-cyclic saturated hydrocarbyl, optionally substituted with one or more substituents; with the proviso that when 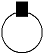
is piperidyl, Z¹ is cyclohexyl, L is —(CHR⁴)ₚ—, and p is 1, then R⁴ is not hydrogen. Particularly preferred compounds of formula (Ia) include those compounds where the nitrogen-containing alicyclic group is piperidyl and Z¹ is cyclooctyl such as compounds SR 14148 and 14150. Other compounds within this formula include Compounds SR 16476, SR 16477 and SR 16501.

Compound (Ib) has the structure: 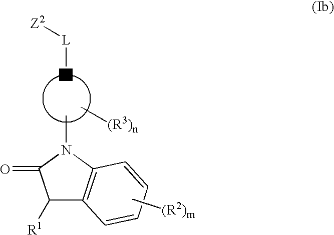
where Z² is a mono-cyclic unsaturated hydrocarbyl, optionally substituted with one or more substituents; with the proviso that when 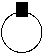
is piperidyl, Z² is phenyl, L is —(CHR⁴)ₚ—, and p is 1, then R⁴ is not hydrogen.

Compound (Ic) has the structure: 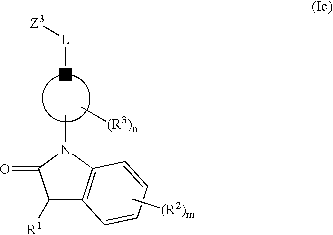
where Z³ is a bi-cyclic hydrocarbyl, optionally substituted with one or more substituents; with the proviso that when 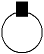
is piperidyl, Z³ is tetrahydronaphthyl or napthyl, L is —(CHR⁴)ₚ—, and p is 1, then R⁴ is not hydrogen. Particular preferred compounds of formula (Ic) include those compounds where the aliphatic nitrogen-containing group is piperidyl, L is —(CHR⁴)ₚ—, p is 0 and Z³ is decahydronaphthyl such as compounds SR 16406 and 16407. Other compounds within this formula include Compounds SR 16405, SR 16439, SR 16408, SR 16432, SR 16433, SR 16490, SR 16434, and SR 16414.

Compound (Id) has the structure: 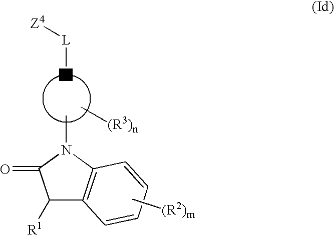
where Z⁴ is a tri-cyclic hydrocarbyl, optionally substituted with one or more substituents. An example of a compound within this formula is SR 16411.

Compound (le) has the structure: 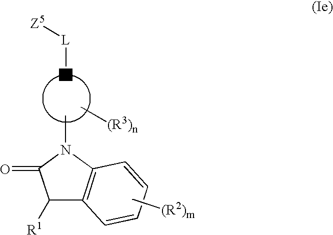
where Z⁵ is hydrogen, and p in the L linker, —(CHR⁴)ₚ—, is an integer from 1-4.

The compounds may be in the form of a pharmacologically active equivalent. Such equivalents include, by way of illustration and not limitation, salts, esters, amides, prodrugs, conjugates, active metabolites, isomers, analogs, or other derivatives, or they may be modified by appending one or more appropriate functionalities to enhance selected biological properties. Such modifications are known in the art and include those that increase biological penetration into a given biological system, increase oral bioavailability, increase solubility to allow administration by injection, and the like. Accordingly, reference to a “compound” or an “active agent” herein is intended to include the compound itself, as well as its pharmacologically active equivalents.

Pharmacologically active equivalents of the active agents may be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992).

For example, acid addition salts of the compounds can be prepared using standard procedures known to those skilled in the art of synthetic organic chemistry. Acid addition salts are prepared from the free base (e.g., compounds having a substituted or unsubstituted amino group or other basic nitrogen-containing functionalities) using conventional means, involving reaction with a suitable acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Preferred acid addition salts of the present compounds are the hydrochloride, tartrate, citrate, fumarate, succinate, benzoate and malonate salts. Conversely, preparation of basic salts of any acidic moieties that may be present may be carried out in a similar manner using a pharmaceutically acceptable base such as sodium hydroxide, potassium hydroxide, ammonium hydroxide, calcium hydroxide, trimethylamine, or the like. Preparation of esters involves reaction of a hydroxyl group with an esterification reagent such as an acid chloride. Amides may be prepared from esters, using suitable amine reactants, or they may be prepared from an anhydride or an acid chloride by reaction with ammonia or a lower alkyl amine. Prodrugs, conjugates, and active metabolites may also be prepared using techniques known to those skilled in the art or described in the pertinent literature. Prodrugs and conjugates are typically prepared by covalent attachment of a moiety that results in a compound that is therapeutically inactive until modified by an individual's metabolic system.

Other derivatives and analogs of the active agents may be prepared using standard techniques known to those skilled in the art of synthetic organic chemistry, or may be deduced by reference to the pertinent literature.

In addition, those novel compounds containing chiral centers can be in the form of a single enantiomer or as a racemic mixture of enantiomers. In some cases, i.e., with regard to certain specific compounds illustrated herein, chirality (i.e., relative stereochemistry) is indicated. In other cases, it is not, and such structures are intended to encompass both the enantiomerically pure form of the compound shown as well as a racemic mixture of enantiomers. Preparation of compounds in enantiomerically form may be carried out using an enantioselective synthesis; alternatively, the enantiomers of a chiral compound obtained in the form of the racemate may be separated post-synthesis, using routine methodology.

III. Utility of the Compounds of the Invention

The compounds of the invention are potent agonists or antagonists of the nociceptin receptor. Without intending to be limiting in any manner, in general, it has been observed that compounds of formula (I) where p is 0 tend to exhibit agonist activity, while compounds where p is 1 tend to exhibit antagonist activity.

In particular, the compounds find utility in a method for modulating a process mediated by the nociceptin receptor by administering to a mammalian subject an amount of a compound of the invention that is effective to modulate the process. Such processes include the in vivo modulation of a drug or alcohol abuse disorder, the in vivo modulation of pain (acute and chronic), and the in vivo modulation of anxiety and stress disorders. The compounds of the invention find particular utility in a method for modulating the nociceptin receptor in a patient in need of such modulation, where a therapeutically effective amount of a compound, for example, the compounds of formula (I), are administered to the patient.

The compounds of the invention can be used to treat a variety of specific conditions, diseases, and disorders that are treatable by modulating the nociceptin receptor. In addition, to the processes noted above, the compounds of the invention have utility in the in vivo modulation of eating disorders, the in vivo modulation of schizophrenia, the in vivo modulation of Parkinsonism, the in vivo modulation of depression, the in vivo modulation of cognitive dysfunction, and the in vivo modulation of cognitive brain function, where modulation is intended to include both the treatment and the prevention of the disease state.

Thus, one preferred embodiment of the invention is a method for treating or preventing eating disorders such as obesity, schizophrenia, Parkinsonism, depression, cognitive dysfunction such as the memory loss associated with Alzheimer's disease or other dementias, or the amelioration of reduced cognitive brain function, comprising administering to an individual in need thereof, a therapeutically effective amount of an N-substituted indolin-2-one compound of the invention.

In addition, the treatment of individuals with a drug or alcohol abuse disorder and the treatment of pain are of particular interest. The use of the compounds as anxiolytics, drugs that are useful in the prevention or treatment of anxiety or stress or mental disorders whose primary feature is anxiety, is also of interest.

Other utilities relate to those disease states outside of the central nervous system that are affected by the endogenous opioid-like peptide, nociceptin, or the receptor to which it binds, the ORL1 receptor. For example, low doses of nociceptin increase the renal excretion of water and decrease urinary sodium excretion which render this compound interesting for the treatment or prevention of hyponatremia (U.S. Pat. No. 5,840,696 to Kapusta et al.), for example hyponatremia that is associated with heart failure or that is associated with intensive diuretic therapy with thiazides and/or loop diuretics. More recently, it has been shown that compounds having nociceptin-like activity or binding activity at the ORL1 receptor exhibit a significant sodium-sparing and potassium-sparing aquaretic effect, which is beneficial in the treatment of edema-forming pathological conditions associated with hyponatremia and/or hypokalemia U.S. Patent Publication No. 20030040472 to Larsen et al.). Accordingly, the compounds of the invention also have utility in methods of treating or preventing: hyponatremia; hypokalemia; a water retaining condition, examples of which include, congestive heart failure, liver cirrhosis, nephrotic syndrome and hypertension; multiple organ failure, for example, acute renal failure; hypertension; and edema, particularly edema that is associated with coronary heart failure.

It will be appreciated by those skilled in the art that numerous other uses of the present compounds are possible as well. Compounds of the invention, referred to below as “test” compounds, can be screened for the desired activity by methods that are well known in the art.

For some uses, it may be preferred that the compound have selectivity for the nociceptin receptor over the other opioid receptors, i.e., the μ, κ, and δ opioid receptors. Selectivity is preferably at least 10-fold, more preferably at least 20-fold, and even more preferably at least 50-fold. However, for other utilities, for example for pain management, it may be desirable to use compounds that also have some degree of affinity for one or more of the opioid receptors. Compounds may be selected for their desired binding specificities and/or affinities using the assays described below.

Test compounds are first tested for binding affinity at ORL1, as shown in Example 7. Affinity is determined using [³H]N/OFQ binding to membranes derived from CHO cells transfected with human ORL1. IC₅₀ values are then determined by the curve fitting program Prism, and Kᵢ values calculated from the formula Kᵢ=IC₅₀/(1+L/K_{d}), where K_{d} is the binding affinity of [³H]N/OFQ and L is the concentration of [³H]N/OFQ used. For test compounds having reasonably high affinity (Kᵢ<1.0 μM), their activity is measured for both stimulation of [³⁵S]GTPγS binding (Example 9) and inhibition of cAMP accumulation (see Example 10) in cells that have been transfected with ORL1. [³⁵S]GTPγS binding has the advantages of being an easier and more reproducible assay as well as providing more facile identification of partial agonist compounds.

To study the activity of antagonists, test compounds that are found to exhibit some binding affinity but little or no agonist activity are tested for their ability to block N/OFQ inhibition of forskolin-stimulated cAMP accumulation and N/OFQ stimulation of [³⁵ ]GTPγS binding in CHO cells transfected with ORL1. Dose response curves for N/OFQ are conducted in the presence of varying concentrations of the antagonist, Schild plots are constructed, and pA₂ values determined. Test compounds are also tested for binding affinity, as well as selectivity versus the μ, δ, and κ opioid receptors, as shown in Example 8.

Once the activity of the test compound is assessed in vitro, the behavioral effects of the compound are readily determined. N/OFQ produces an increase in locomotor activity and is antinociceptive spinally and pronociceptive supraspinally (Calo et al. (2000) Br. J. Pharmacol. 129:1261-1283). Various doses of test compounds are administered subcutaneously (SC) to mice. The effect on locomotor activity is determined using automated behavioral activity monitors or the ability to increase or decrease tail flick latencies in mice is tested (See Example 11).

The rewarding/aversive properties of the test compounds after systemic administration in mice are determined using the place conditioning (PC) paradigm, which offers several advantages (see Example 13). First, both the rewarding and aversive properties of drugs is assessed using this procedure. Second, other behavioral measures such as locomotor activity are assessed following acute as well as repeated drug administration. Third, testing is done under drug-free conditions. This is a major advantage, since motor effects of many drugs can obscure measurements of their rewarding and aversive effects when other methods are used. Finally, this method allows for controlled drug doses, whereas with the self-administration paradigm the dose administered is dependent on the animal's rate of responding. Indices of locomotion and exploratory behavior are also obtained using this paradigm.

Test compounds may be evaluated on a chronic pain model, which uses rats whose sciatic nerve on one leg has been ligated. This model, referred to as the “Bennett Chronic Constriction Injury” model, produces chronic pain and allodynia in the affected foot (Bennett et al. (1988) Pain 33:87-107). Foot withdrawal latency is determined, and the effect of systemic administration of the agonist and antagonist test compounds is then determined (See Example 11). Since N/OFQ appears to have differing actions subsequent to intracerebroventricular (ICV) and intrathecal (IT) administration, studies on the systemic administration of test compounds are very important.

The test compounds are also evaluated, in conjunction with investigating the interaction of ORL1 and opioid receptors, using various behavioral techniques and opioid receptor knock-out mice. Interactions between the ORL1 and opiate system are evident, since N/OFQ reverses opioid-mediated stress-induced antinociception as well as morphine-induced analgesia and inhibits morphine withdrawal (Kotlinska et al., supra). In addition, N/OFQ attenuates the rewarding effects of morphine in the PC paradigm (Murphy et al. (1999) Brain Res. 832:168-170), and tolerance to morphine antinociception is reduced in ORL1 knockout mice (Ueda et al. (1997) Neurosci. Lett. 237:136-138). Thus, examining the interaction between the test compounds and opioid receptors, will provide valuable insight. This interaction is tested in regard to a variety of opioid actions, including antinociception, tolerance development, and reward. The test compounds are evaluated for their ability to block antinociception induced by SC administration of morphine and by ICV administration of known μ-, δ-, and κ-selective opiates. Appropriate time- and dose-response curves for both all compounds is then determined, and the mice observed for behavioral changes such as an increase or decrease in locomotor activity. Compounds are also tested to examine whether they potentiate opiate-induced analgesia. For these experiments, various concentrations of the test compound are administered concurrently with subanalgesic doses of morphine. Tail flick latency is then determined as described herein.

Since it is believed that ORL1 ligands alter opioid agonist-induced behavior by their effects on the opioid system, knock-out mice lacking μ-opioid receptors are useful to evaluate these compounds. Antagonist test compounds are injected concurrently with morphine to evaluate whether they have the ability to attenuate morphine tolerance development. Tolerance is developed by a single daily injection of morphine (5 mg/kg), in the presence or absence of the antagonist test compound. Percent analgesia is determined daily using the tail flick assay over a period of up to 12 days, as described in Kolesnikov et al. (1992) Eur. J. Pharmacol. 221:399-400). Mice lacking μ receptors can be used to determine the extent of interaction of ORL1 receptors with the opioid receptor system in regard to the decrease in the analgesic effects of opiates. Thus, ORL1 and opiate ligands are co-administered in μ receptor knock-out mice and tail flick latency be measured. In addition, other behavioral changes are also observed.

Test compounds can also be for their ability to alter opiate-induced place preference. Mice are assigned to groups receiving SC injections of saline or 3 mg/kg morphine, a dose previously shown to produce a conditioned place preference (Belzung et al. (2000) Pharmacol. Biochem. Behav. 65:419-423), co-administered with one of four doses of a test compound. Active doses of the test compounds as well as the time course of action are determined from initial experiments.

The effects of test compounds on the rewarding properties of direct and indirect dopamine agonists can be investigated. Previous studies have shown that N/OFQ and other the synthetic ORL1 agonists seem to be devoid of rewarding properties on their own (Devine et al. (1996) Neurochem. Res. 21:1387-1396; Jenck et al. (1997) Proc. Natl. Acad. Sci. USA 94:14854-14858), whereas N/OFQ attenuates the rewarding properties of ethanol and morphine (Calo et al., supra). The mesolimbic dopamine system is an important component in modulating behaviors elicited by drugs of abuse, and stimulation of ORL1 receptors has been shown to modulate mesolimbic dopamine activity. Administration of N/OFQ decreases dopamine release in the nucleus accumbens of anesthetized rats (Murphy et al. (1996) Neuroscience 75:1-4). Thus, full examination of the interaction of ORL1 and dopaminergic receptors should provide insight into the therapeutic utility of the test compounds in the treatment of drug abuse. Specifically, the interaction of the test compounds with the drugs of abuse cocaine and amphetamine (indirect dopamine agonists) as well as with the direct dopamine agonist apomorphine are examined. The effects of test compounds on cocaine, amphetamine, and apomorphine induced place preference and behaviors is assessed as follows. Mice are assigned to groups receiving SC injections of saline, 10 mg/kg cocaine, 1 mg/kg amphetamine, or 2 mg/kg apomorphine (Belzung et al., supra) co-administered with one of four doses of a test compound. Active doses of the test compound as well as the time course of action is determined from initial experiments.

IV. Administration of the Compounds of the Invention

The compounds of the invention may be conveniently formulated into pharmaceutical compositions composed of one or more of the compounds in association with a pharmaceutically acceptable carrier. See Remington: The Science and Practice of Pharmacy, 20^{th} edition (Lippincott Williams & Wilkins, 2000) and Ansel et al., Pharmaceutical Dosage Forms and Drug Delivery Systems, 6^{th} Ed. (Media, P A: Williams & Wilkins, 1995), which disclose typical carriers and methods for preparing pharmaceutical compositions, which methods may be used as described or modified to prepare pharmaceutical formulations and dosage forms containing a compound of the invention.

Accordingly, one embodiment of the invention is a pharmaceutical composition comprising a therapeutically effective amount of a compound of the invention in combination with a pharmaceutically acceptable carrier.

The compounds of the invention may be administered orally, parenterally, rectally, vaginally, buccally, sublingually, nasally, by inhalation, topically, transdermally, or via an implanted reservoir in dosage forms containing conventional non-toxic pharmaceutically acceptable carriers and excipients. The term “parenteral” as used herein is intended to include subcutaneous, intravenous, and intramuscular injection.

The amount of the compound administered will, of course, be dependent the condition or disorder being treated. For example, the compounds of the invention find utility as a reliever against tolerance to or dependence on a narcotic analgesic represented by morphine, an analgesic or an analgesic enhancer, an anxiolytic, an antiobestic, a remedy for schizophrenia, a remedy for Parkinsonism, an antidepressant, and a drug for ameliorating brain finction.

In addition, the amount of the compound administered will also be dependent on the particular active agent, the condition or disorder being treated, the severity of the condition or disorder, the subject's individual traits (e.g., sex, age, weight), the mode of administration and other pertinent factors known to the prescribing physician. Generally, however, dosage will be in the range of approximately 0.001 mg/kg/day to 100 mg/kg/day, more preferably in the range of approximately 0.01 to 20 mg/kg/day, and most preferably in the range of about 0.1 mg/kg/day to 10 mg/kg/day.

Depending on the intended mode of administration, the pharmaceutical formulation may be a solid, semi-solid or liquid, such as, for example, a tablet, a capsule, caplets, a liquid, a suspension, an emulsion, a suppository, granules, pellets, beads, a powder, or the like, preferably in unit dosage form suitable for single administration of a precise dosage. Suitable pharmaceutical compositions and dosage forms may be prepared using conventional methods known to those in the field of pharmaceutical formulation.

For those compounds that are orally active, oral dosage forms are generally preferred, and include tablets, capsules, caplets, and nonaqueous solutions, suspensions and or syrups, and may also comprise a plurality of granules, beads, powders or pellets that may or may not be encapsulated. Preferred oral dosage forms are tablets and capsules.

Tablets may be manufactured using standard tablet processing procedures and equipment. Direct compression and granulation techniques are preferred. In addition to the active agent, tablets will generally contain inactive, pharmaceutically acceptable carrier materials such as binders, lubricants, disintegrants, fillers, stabilizers, surfactants, coloring agents, and the like. Binders are used to impart cohesive qualities to a tablet, and thus ensure that the tablet remains intact. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum. Lubricants are used to facilitate tablet manufacture, promoting powder flow and preventing particle capping (i.e., particle breakage) when pressure is relieved. Useful lubricants are magnesium stearate, calcium stearate, and stearic acid. Disintegrants are used to facilitate disintegration of the tablet, and are generally starches, clays, celluloses, algins, gums, or crosslinked polymers. Fillers include, for example, materials such as silicon dioxide, titanium dioxide, alumina, talc, kaolin, powdered cellulose, and microcrystalline cellulose, as well as soluble materials such as mannitol, urea, sucrose, lactose, dextrose, sodium chloride, and sorbitol. Stabilizers, as well known in the art, are used to inhibit or retard drug decomposition reactions that include, by way of example, oxidative reactions.

Capsules are also preferred oral dosage forms, in which case the active agent-containing composition may be encapsulated in the form of a liquid or solid (including particulates such as granules, beads, powders or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. Materials and methods for preparing encapsulated pharmaceuticals are well known to those in the field of pharmaceutical formulation.

Oral dosage forms, whether tablets, capsules, caplets, or particulates, may, if desired, be formulated so as to provide for gradual, sustained release of the active agent over an extended time period. Generally, as will be appreciated by those of ordinary skill in the art, sustained release dosage forms are formulated by dispersing the active agent within a matrix of a gradually hydrolyzable material such as an insoluble plastic (e.g., polyvinyl chloride or polyethylene), or a hydrophilic polymer, or by coating a solid, drug-containing dosage form with such a material. Hydrophilic polymers useful for providing a sustained release coating or matrix include, by way of example: cellulosic polymers such as hydroxypropyl cellulose, hydroxyethyl cellulose, hydroxypropyl methyl cellulose, methyl cellulose, ethyl cellulose, cellulose acetate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate; and vinyl polymers and copolymers such as polyvinyl pyrrolidone, polyvinyl acetate, and ethylene-vinyl acetate copolymer.

Preparations according to this invention for parenteral administration include sterile nonaqueous solutions, suspensions, and emulsions. Examples of nonaqueous solvents or vehicles are propylene glycol, polyethylene glycol, vegetable oils, such as olive oil and corn oil, gelatin, and injectable organic esters such as ethyl oleate. Parenteral formulations may also contain adjuvants such as preserving, wetting, emulsifying, and dispersing agents. The formulations are rendered sterile by incorporation of a sterilizing agent, filtration through a bacteria-retaining filter, irradiation, or heat. They can also be manufactured using a sterile injectable medium.

The compounds of the invention may also be administered through the skin or mucosal tissue using conventional transdermal drug delivery systems, wherein the active agent is contained within a laminated structure that serves as a drug delivery device to be affixed to the skin. In such a structure, the drug composition is contained in a layer, or “reservoir,” underlying an upper backing layer. The laminated structure may contain a single reservoir, or it may contain multiple reservoirs. In one embodiment, the reservoir comprises a polymeric matrix of a pharmaceutically acceptable contact adhesive material that serves to affix the system to the skin during drug delivery. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. Transdermal drug delivery systems may in addition contain a skin permeation enhancer.

Although the present compositions will generally be administered orally, parenterally, or transdermally, other modes of administration are suitable as well. For example, administration may be rectal or vaginal, preferably using a suppository that contains, in addition to the active agent, excipients such cocoa butter or a suppository wax. Formulations for nasal or sublingual administration are also prepared with standard excipients well known in the art. The pharmaceutical compositions of the invention may also be formulated for inhalation, e.g., as a solution in saline, as a dry powder, or as an aerosol. Transdermal administration is also a suitable delivery route for compounds of the invention.

V. Synthesis of the Compounds of the Invention

The compounds of the invention may be prepared in high yield using relatively simple, straightforward methods as exemplified in the experimental section herein. Syntheses of representative compounds are detailed in the Examples.

All patents, publications, and other published documents mentioned or referred to herein are incorporated by reference in their entireties.

It is to be understood that while the invention has been described in conjunction with the preferred specific embodiments thereof, that the foregoing description as well as the examples that follow, are intended to illustrate and not limit the scope of the invention. It should be understood by those skilled in the art that various changes may be made and equivalents may be substituted without departing from the scope of the invention, and further that other aspects, advantages and modifications will be apparent to those skilled in the art to which the invention pertains.

### Experimental

The practice of the present invention will employ, unless otherwise indicated, conventional techniques of synthetic organic chemistry, biological testing, and the like, which are within the skill of the art. Such techniques are explained fully in the literature. See, for example, Kirk-Othmer's Encyclopedia of Chemical Technology; and House's Modern Synthetic Reactions. Most ligands were synthesized by reductive amination of the appropriate aldehyde or ketone with the common intermediate N-1-(4-piperidinyl)-1,3-dihydroindol-2-one (5), which was obtained from SR 16412 by debenzylation. Reductive amination is described in Abdel-Magid et al. (1996) J. Org. Chem. 61:3849-3862, and the conversion of ketones to amines is described in U.S. Pat. No. 6,258,825 to Ozaki et al.

In the following examples, efforts have been made to insure accuracy with respect to numbers used (e.g., amounts, temperature, etc.) but some experimental error and deviation should be accounted for. Unless indicated otherwise, temperature is in degrees C and pressure is at or near atmospheric. All solvents were purchased as HPLC grade, and all reactions were routinely conducted under an inert atmosphere of argon unless otherwise indicated. All reagents were obtained commercially unless otherwise indicated. Most aldehydes and ketones used were commercially available or could be easily accessed by Swern oxidation of their hydroxyl precursors.

ABBREVIATIONS

| | |
|---|---|
| AcOH | acetic acid |
| Bn | benzyl |
| DCE | 1,2-dichloroethane |
| DCM | dichloromethane |
| DMF-THF | N,N-Dimethylformamide/tetrahydrofuran |
| EtOAc | ethyl acetate |
| EtOH | ethanol |
| MeI | iodomethane |
| MeOH | methanol |
| NaBH(OAc)₃ | sodium triacetoxyborohydride |
| Pd-C | palladium on activated carbon |
| p-TsOH | p-toluenesulfonic acid monohydrate |
| RT | room temperature |
| tBu | tert-butyl |

### EXAMPLE 1

### Synthesis of Compounds of Formula (5)

Compound (5) was prepared using N-benzyl piperidone as the starting material, as described in U.S. Pat. No. 3,325,499 to Poos:

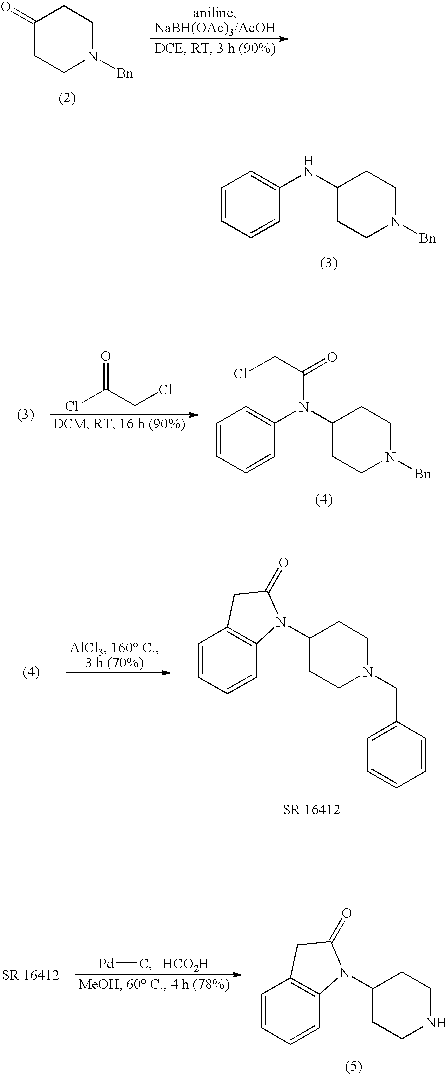

For the debenzylation of SR 16412: To a 100 mL round-bottom flask, were added compound SR 16412 (1.50 g, 4.9 mmol), Pd—C (wt. 10%, 0.70 g), formic acid (96%, 1.6 mL, 42.4 mmol), and anhydrous methanol (40 mL). This reaction mixture was stirred at 60° C. for 4 h. The solvent removed under vacuum, the residue basified with IN NaOH_{(aq)} to pH>12, and then extracted with dichloromethane. The extract was washed with brine, dried over anhydrous Na₂SO₄, and evaporated to afford a solid (5) (0.82 g, yield: 78%).

### EXAMPLE 2

### Large Scale Synthesis of Compounds of Formula (5)

For larger scale preparations of compounds of formula (5), di-tert-butylmalonate (6) was used as the starting material, as described in Forbes (2001) Tetrahedron Letters 2:6943-6945:

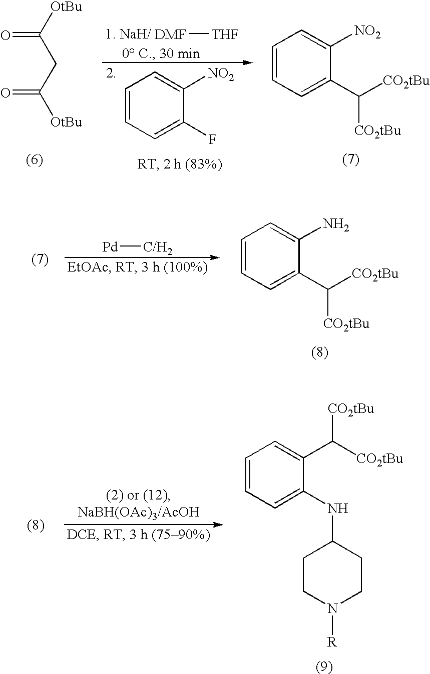

Compound (8), N-benzyl piperidone, and sodium triacetoxyborohydride were then mixed in a ratio of 1.1/1/1.5 in 1,2-dichloroethane and then 1 equivalent of acetic acid added. The mixture was stirred under argon at room temperature for 16 h and treated with 1N NaOH to pH>12. The expected product was then extracted with ethyl acetate. The extract was washed with brine, dried over anhydrous Na₂SO₄, and evaporated to give crude product (9; R=benzyl), which was purified by flash chromatography on silica gel (yield: 75%).

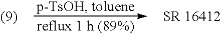

Compound (9) underwent decarboxylative cyclization to yield compound SR 16412. Compound (9) and p-toluenesulfonic acid monohydrate were mixed in a 1 to 2 molar ratio in toluene (10 mL per mmol of (9)). With a Dean-Stark trap, the reaction mixture was refluxed for 1 h. Solvent was removed under vacuum, the residue basified with 1N NaOH, and then partitioned with ethyl acetate. The organic phase was separated, washed with brine, dried over anhydrous Na₂SO₄, and evaporated to give crude product, which was, in most cases, pure by TLC. Analytical samples were obtained by flash chromatography on silica gel (yield: 89%).

Analytical data for compound SR 16412: MS: (ESI+) m/e [M+H] 307.10. ¹H (CDCl₃): δ 7.37-7.08 (m, 8H), 6.96 (t, J=7.3 Hz, 1H), 4.29 (tt, J=12.6, 4.4 Hz, 1H), 3.51 (s, 2H), 3.43 (s, 2H), 2.98 (d, J=11.4 Hz, 2H), 2.45 (qd, J=12.4, 3.8 Hz, 2H), 2.10 (td, J=11.9, 2.1 Hz, 2H), 1.62 (m, 2H). ¹³C{¹H} (CDCl₃): δ 175.0 (1C), 144.1 (1C), 138.7 (1C), 129.3 (2C), 128.5 (2C), 127.8 (1C), 127.3 (1C), 125.1 (1C), 124.8 (1C), 122.0 (1C), 110.5 (1C), 63.2 (1C), 53.4 (2C), 50.3 (1C), 36.1 (1C), 28.4 (2C). Cald. (C₂₀H₂₂N₂O) %: C, 78.40; H, 7.24; N, 9.14. Found %: C, 78.28; H, 7.31; N, 9.20.

SR 16412 was then debenzylated as described in Example 1.

### EXAMPLE 3

### Reductive Amination of Compound (5)

Ketones used in this reaction were commercially available or were readily synthesized by known literature methods. Aldehydes were obtained via Swem oxidation of the respective hydroxymethyl compounds, which were commercially available. 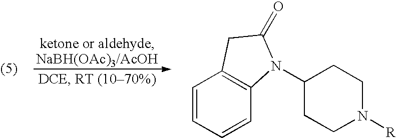
where the R groups were:

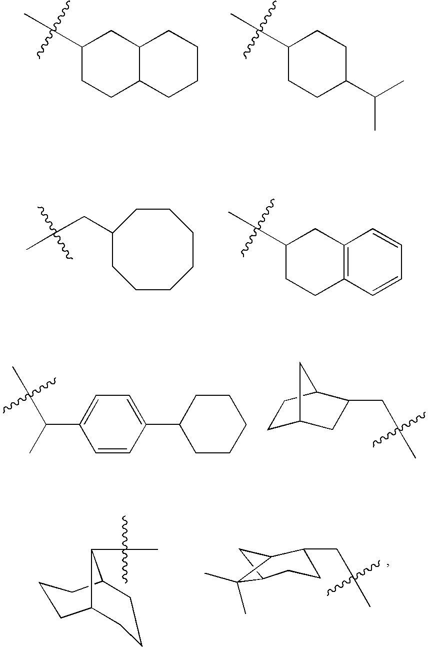

Compound (5) underwent reductive amination with aldehydes and steric unhindered ketones to produce compounds SR 14148, SR 16405, SR 16406, SR 16407, SR 16410, SR 16432, SR 16433, SR 16434, SR 16476, SR 16477, and SR 16414. Compound (5) (1 mmol), a selected aldehyde or ketone (1 mmol), sodium triacetoxyborohydride (1.5 mmol), and acetic acid (1 mmol) were mixed in 1,2-dichloroethane (5 niL), and the mixture stirred under argon at room temperature for 2 h (for aldehydes) or 12-16 h (for ketones). The reaction mixture was treated with 1N NaOH to pH>12, and extracted with ethyl acetate. The extract was washed with brine, dried over anhydrous Na₂SO₄, and evaporated to give crude product, which was purified by flash chromatography on silica gel (yield: 10-70%).

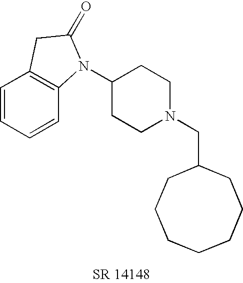

Analytical data for compound SR 14148: MS: (ESI+) m/e (M+H) 341. ¹H NMR (300 MHz, CDCl₃): δ 7.29-7.20 (m, 2H), 7.19-7.12 (m, 1H), 7.04-6.96 (m, 1H), 4.30 (tt, J=12.6, 4.5 Hz, 1H), 3.50 (s, 2H), 2.99 (d, J=11.4 Hz, 2H), 2.43 (qd, J=12.3, 3.6 Hz, 2H), 2.16-1.98 (m, 3H), 1.78-1.40 (m, 16H), 1.30-1.15 (m, 2H). Anal. calcd. for C₂₂H₃₃ClN₂O (hydrochloride salt): C, 70.10; H, 8.82; N, 7.43; Cl, 9.41. Found: C, 69.84; H, 8.80; N, 7.38; Cl, 9.62.

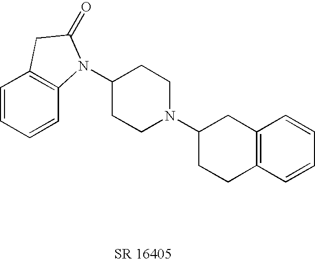

Analytical data for compound SR 16405: MS: (APCI+) m/e [M+H] 347.28. ¹H (CDCl₃): δ 7.27-6.96 (m, 8H), 4.44-4.26 (m, 1H), 3.50 (s, 2H), 3.20-3.06 (m, 2H), 3.00-2.74 (m, 5H), 2.56-2.36 (m, 4H), 2.18-2.04 (m, 1H), 1.82-1.58 (m, 3H). ¹³C{¹H} (CDCl₃): δ 175.1 (1C), 144.0 (1C), 136.6 (1C), 136.3 (1C), 129.7 (1C), 128.8 (1C), 127.8 (1C), 126.0 (1C), 125.9 (1C), 125.1 (1C), 124.8 (1C), 122.0 (1C), 110.8 (1C), 60.7 (1C), 50.5 (1C), 49.5 (1C), 48.9 (1C), 36.1 (1C), 31.8 (1C), 29.9 (1C), 28.9 (2C), 26.5 (1C). Cald. (C₂₃H₂₆N₂O. 0.1H₂O) %: C, 79.32; H, 7.58; N, 8.04. Found %: C, 79.21; H, 7.61; N, 8.05.

The SR 16406 and SR 16407 cis and trans isomers were separated by chromatography.

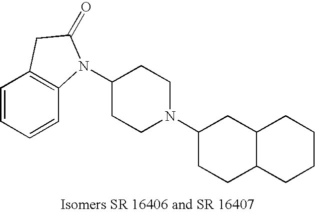

Analytical data for compound SR 16406: MS: HRMS m/e [M+H]: Cald. (C-₂₃H₃₂N₂O): 353.2593. Found: 353.2587. ¹H (CDCl₃): δ 7.30-7.22 (m, 2H), 7.13 (d, J=7.6 Hz, 1H), 7.01 (t, J=7.3 Hz, 1H), 4.25 (tt, J=12.6, 4.4 Hz, 1H), 3.51 (s, 2H), 3.32-3.18 (m, 2H), 2.42 (qt, J=12.6, 3.3 Hz, 2H), 2.29 (br, 1H), 2.10-0.80 (m, 20H). ¹³C{¹H} (CDCl₃): δ 175.2 (1C), 144.3 (1C), 127.8 (1C), 125.1 (1C), 124.7 (1C), 122.0 (1C), 110.5 (1C), 58.9 (1C), 50.9 (1C), 50.6 (1C), 50.4 (1C), 44.7 (1C), 37.1 (1C), 37.0 (1C), 36.2 (1C), 34.3 (1C), 34.0 (1C), 29.5 (1C), 28.6 (1C), 28.5 (1C), 28.3 (1C), 27.0 (1C), 26.9 (1C).

Analytical data for compound SR 16407 (a mixture of isomers): HRMS m/e [M+H]: Cald. (C₂₃H₃₂N₂O): 353.2593. Found: 353.2593.

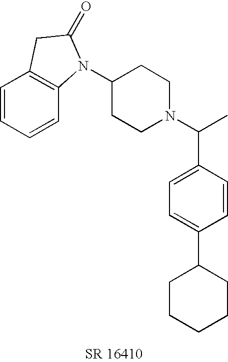

Analytical data for compound SR 16410: HRMS m/e [M+H]: Cald. (C₂₇H₃₄N₂O): 403.2749. Found: 403.2750. ¹H (CDCl₃): δ 7.28-7.16 (m, 7H), 7.00 (td, J=7.4, 1.2 Hz, 1H), 4.24 (tt, J=12.6, 4.4 Hz, 1H), 3.49 (s, 2H), 3.56-3.40 (m, 1H), 3.20-2.92 (m, 2H), 2.58-1.30 (m, 20H). ¹³C{¹H} (CDCl₃): δ 175.1 (1C), 146.9 (1C), 144.1 (1C), 140.9 (1C), 127.7 (3C), 126.7 (2C), 125.1 (1C), 124.7 (1C), 122.0 (1C), 110.7 (1C), 64.2 (1C), 50.5 (1C), 50.3 (2C), 44.4 (1C), 36.1 (1C), 34.7 (2C), 28.7 (2C), 27.2 (2C), 26.4 (1C), 19.5 (1C).

The endo and exo isomers SR 16432 and SR 16433 were separated by chromatography and analyzed separately.

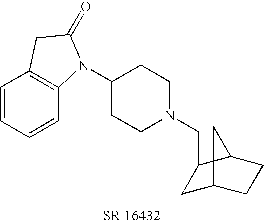

Analytical data for compound SR 16432: MS: (ESI+) m/e [M+H] 325. ¹H NMR (300 MHz, CDCl₃) δ 7.28-7.13 (m, 3H), 7.00 (td, J=7.5, 0.9 Hz, 1H), 4.31 (tt, J=12.3, 4.2 Hz, 1H), 3.49 (s, 2H), 3.03 (t, 10.9 Hz, 2H), 2.44 (qd, J=12.0, 2.7 Hz, 2H), 2.30-2.17 (m, 2H), 2.16-1.98 (m, 4H), 1.72-1.05 (m, 11H). HRMS: calcd. for C₂₁H₂₉N₂O 325.228, found: 325.2291.

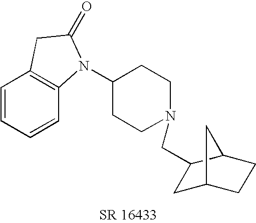

Analytical data for compound SR 16433: MS: (ESI+) m/e [M+H] 325. ¹H NMR (300 MHz, CDCl₃) δ 7.27-7.13 (m, 3H), 7.00 (td, J=7.2, 1.5 Hz, 1H), 4.30 (tt, J=12.9, 4.4 Hz, 1H), 3.50 (s, 2H), 3.05 (t, J=12.9 Hz, 2H), 2.55-1.88 (m, 8H), 1.84-1.59 (m, 4H), 1.57-1.43 (m, 2H), 1.43-1.20 (m, 3H), 1.16-1.02 (m, 1H), 0.69 (ddd, J=2.4, 5.1, 12.3 Hz, 1H). HRMS: calcd. for C₂₁H₂₉N₂O 325.228, found: 325.2263.

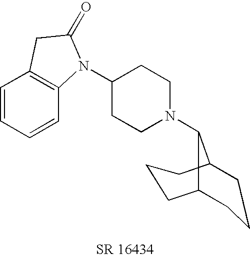

Analytical data for compound SR 16434: MS: (ESI+) m/e [M+H] 339. ¹H NMR (300 MHz, CDCl₃) δ 7.29-7.22 (m, 2H), 7.14 (d, J=7.5 Hz, 1H), 7.01 (dt, J=1.0, 7.2 Hz, 1H), 4.29 (tt, J=12.6, 3.9 Hz, 1H), 3.50 (s, 2H), 3.28 (d, J=11.7 Hz, 2H), 2.42 (qd, J=12.3, 3.3 Hz, 2H), 2.10-1.31 (m, 19H). Anal. calcd. for C₂₂H₃₀N₂O: C, 78.06; H, 8.93; N, 8.27. Found: C, 78.01; H, 9.09; N, 8.27.

The SR 16476 and SR 16477 cis and trans isomers were separated by chromatography.

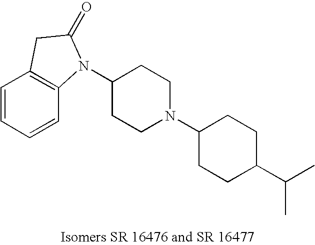

Analytical data for compound SR 16476: MS: (ESI+) m/e [M+H] 341.21. ¹H (CDCl₃): δ 7.30-7.13 (m, 3H), 6.99 (td, J=7.3, 1.2 Hz, 1H), 4.30 (tt, J=12.5, 4.4 Hz, 1H), 3.49 (s, 2H), 3.13 (d, J=11.5 Hz, 2H), 2.41(qd, J=12.3, 3.7 Hz, 2H), 2.36-2.25 (m, 1H), 2.18 (td, J=11.7, 2.1 Hz, 2H), 1.77-1.30 (m, 11H), 1.20-1.06 (m, 1H), 0.89 (d, J=6.7 Hz, 6H). ¹³C{¹H} (CDCl₃): δ 175.0 (1C), 144.1 (1C), 127.7 (1C), 125.0 (1C), 124.7 (1C), 121.9 (1C), 110.7 (1C), 61.7 (1C), 50.6 (1C), 49.6 (2C), 42.0 (1C), 36.1 (1C), 28.8 (3C), 26.5 (2C), 26.0 (2C), 20.9 (2C). Cald. (C₂₂H₃₂N₂O. 0.3H₂O) %: C, 76.39; H, 9.50; N, 8.10. Found %: C, 76.46; H, 9.46; N, 8.02.

Analytical data for compound SR 16477: MS: (ESI+) m/e [M+H] 341.22. ¹H (CDCl₃): δ 7.26-7.15 (m, 3H), 7.05-6.95 (m, 1H), 4.40-4.22 (m, 1H), 3.50 (s, 2H), 3.13-2.95 (m, 2H), 2.50-2.25 (m, 5H), 1.98-1.62 (m, 6H), 1.50-0.94 (m, 6H), 0.86 (d, J=7.0 Hz, 6H). ¹³C{¹H} (CDCl₃): δ 175.0 (1C), 144.0 (1C), 127.7 (1C), 125.0 (1C), 124.7 (1C), 121.9 (1C), 110.8 (1C), 64.2 (1C), 50.6 (1C), 49.2 (2C), 44.2 (1C), 36.1 (1C), 32.9 (1C), 29.4 (2C), 29.0 (2C), 28.8 (2C), 20.1 (2C). Cald. (C₂₂H₃₂N₂O) %: C, 77.60; H, 9.47; N, 8.23. Found %: C, 77.52; H, 9.64; N, 8.31.

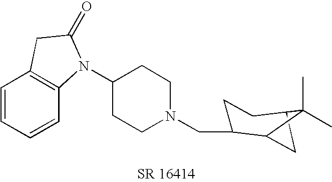

Analytical data for compound SR 16414: MS: (APCI+) m/e [M+H] 353. ¹H (CDCl₃): δ 7.27-7.20 (m, 2H), 7.17-7.12 (m, 1H), 7.00 (td, J=7.2, 1.2 Hz, 1H), 4.29 (tt, J=12.3, 4.1 Hz, 1H), 3.50 (s, 2H), 2.98 (m, 2H), 2.41 (m, 2H), 2.18-1.56 (m, 15H), 1.40-1.24 (m 2H), 1.23 (s, 3H), 0.85 (s, 3H), 0.85 (s, 3H). Cald. (C₂₃H₃₂N₂O) %: C, 78.37; H, 9.15; N, 7.95. Found %: C, 78.22; H, 9.41; N, 7.81.

### EXAMPLE 4

### Synthesis of Compounds of Formula (12)

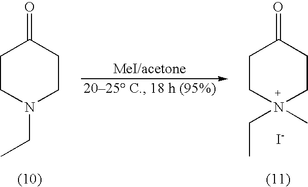

The synthesis of compound (11) is described by Kolczewski et al. (2003) J. Med. Chem. 46:255-264. 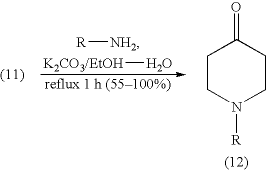
where R was one of the following:

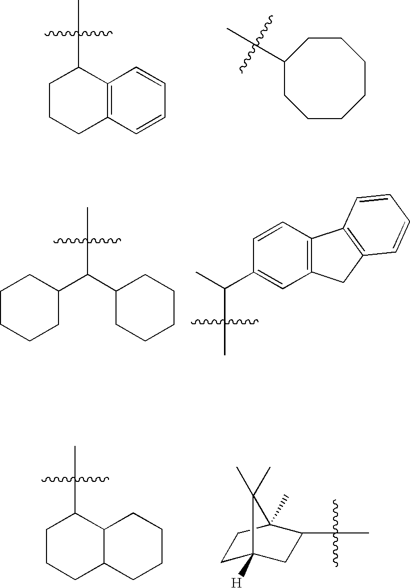

### EXAMPLE 5

Similar to the scheme described in Example 2, Compound (8) was condensed with the desired compound (12) from Example 4 (instead of N-benzyl piperidone), to produce compound (9) under similar reaction conditions (t=12-16 hr). Compound (9) then underwent decarboxylative cyclization, under similar conditions as in Example 2, to yield the compounds SR 14150, SR 16439, SR 16408, SR 16411, SR 16477, and SR 16501. Analytical samples were obtained by flash chromatography on silica gel (yield: 70-90%).

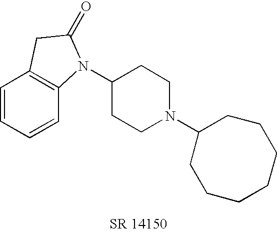

Analytical data for compound SR 14150: MS: (APCI+) m/e [M+H] 327.27. ¹H (CDCl₃): δ 7.25-7.16 (m, 3H), 7.03-6.95 (m, 1H), 4.29 (tt, J=12.6, 4.4 Hz, 1H), 3.49 (s, 2H), 2.90 (d, J=7.2 Hz, 2H), 2.64 (br, 1H), 2.48-2.30 (m, 4H), 1.84-1.40 (m, 16H). ¹³C{¹H} (CDCl₃): δ 175.1 (1C), 144.0 (1C), 127.7 (1C), 125.0 (1C), 124.7 (1C), 122.0 (1C), 110.8 (1C), 64.2 (1C), 50.6 (1C), 48.6 (2C), 36.1 (1C), 29.8 (2C), 29.0 (2C), 26.9 (2C), 26.8 (1C), 26.0 (2C). Cald. (C₂₁H₃₀N₂O) %: C, 77.26; H, 9.26; N, 8.58. Found %: C, 77.15; H, 9.40; N, 8.63.

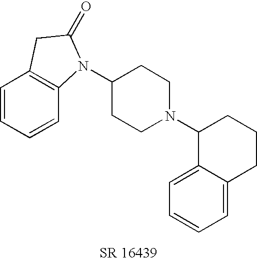

Analytical data for compound SR 16439: MS: (ESI+) m/e [M+H] 347.24. ¹H (CDCl₃): δ 7.78 (d, J=8.4 Hz, 1H), 7.30-6.92 (m, 7H), 4.28 (tt, J=12.3, 4.5Hz, 1H), 3.89 (br, 1H), 3.44 (s, 2H), 3.02-1.50 (m, 14H). ¹³C{¹H} (CDCl₃): δ 175.0 (1C), 144.2 (1C), 138.6 (1C), 138.4 (1C), 129.1 (1C), 128.1 (1C), 127.8 (1C), 126.6 (1C), 126.1 (1C), 125.2 (1C), 124.8 (1C), 122.0 (1C), 110.6 (1C), 63.4 (1C), 51.8 (1C), 50.8 (1C), 45.6 (1C), 36.2 (1C), 30.1 (1C), 29.4 (1C), 29.0 (1C), 22.3 (1C), 21.8 (1C). Cald. (C₂₃H₂₆N₂O) %: C, 79.73; H, 7.56; N, 8.09. Found %: C, 79.56; H, 7.62; N, 8.10.

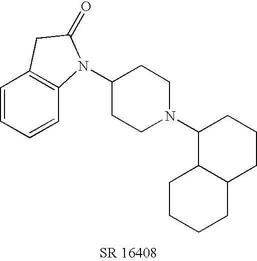

Analytical data for compound SR 16408: MS: (APCI+) m/e [M+H] 353.29. ¹H (CDCl₃): δ 7.27-7.14 (m, 3H), 7.00 (td, J=7.3, 1.2 Hz, 1H), 4.32 (tt, J=12.6, 4.3 Hz, 1H), 3.50 (s, 2H), 3.32-2.98 (m, 2H), 2.54-1.06 (m, 23H). ¹³C{¹H} (CDCl₃): δ 175.1 (1C), 144.1 (1C), 127.7 (1C), 125.0 (1C), 124.7 (1C), 122.0 (1C), 110.8 (1C), 66.7 (1C), 50.7 (1C), 50.5 (1C), 50.4 (1C), 39.5 (1C), 37.0 (1C), 36.1 (1C), 32.8 (1C), 28.8 (1C), 28.6 (1C), 27.1 (1C), 25.9 (1C), 25.3 (1 C), 24.6 (IC), 21.8 (1C), 20.6 (1C). Cald. (C₂₃H₃₂N₂O) %: C, 78.36; H, 9.15; N, 7.95. Found %: C, 78.23; H, 9.18; N, 7.90.

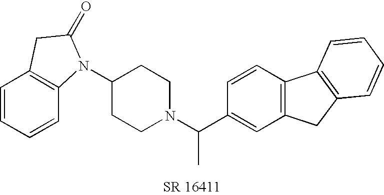

Analytical data for compound SR 16411: MS: (ESI+) m/e [M+H] 409.10. ¹H (CDCl₃): δ 7.75 (t, J=7.9 Hz, 2H), 7.58-7.12 (m, 8H), 7.00 (t, J=7.3 Hz, 1H), 4.24 (tt, J=12.5, 4.4 Hz, 1H), 3.91 (s, 2H), 3.55 (q, J=6.7 Hz, 1H), 3.49 (s, 2H), 3.30-2.96 (m, 2H), 2.60-2.32 (m, 2H), 2.16 (td, J=11.7, 2.1 Hz, 1H), 2.05 (td, J=11.7, 2.1 Hz, 1H), 1.84-1.56 (m, 2H), 1.45 (d, J=6.7 Hz, 3H). ¹³C{¹H} (CDCl₃): δ 175.1 (1C), 144.1 (1C), 143.6 (1C), 143.5 (1C), 143.0 (1C), 141.8 (1C), 140.9 (1C), 127.7 (1C), 126.9 (1C), 126.7 (1C), 126.5 (1C), 125.2 (1C), 125.1 (1C), 124.7 (1C), 124.4 (1C), 122.0 (1C), 119.9 (1C), 119.7 (1C), 110.5 (1C), 64.9 (1C), 50.9 (1C), 50.6 (1C), 50.3 (1C), 37.2 (1C), 36.2 (1C), 28.7 (1C), 28.6 (1C), 20.1 (1C). Cald. (C₂₈H₂₈N₂O) %: C, 82.32; H, 6.91; N, 6.86. Found %: C, 81.95; H, 6.84; N, 6.82.

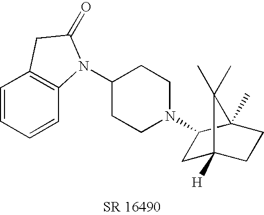

Analytical data for compound SR 16490: MS: (ESI+) m/e [M+H] 353.18. ¹H (CDCl₃): δ 7.26-6.92 (m, 4H), 4.23 (tt, J=12.5, 4.3 Hz, 1H), 3.44 (s, 2H), 3.24-3.02 (m, 2H), 2.50-2.20 (m, 3H), 2.10-1.85 (m, 4H), 1.80-1.50 (m, 4H), 1.35-1.16 (m, 2H), 1.05 (dd, J=12.5, 4.0 Hz, 1H), 0.95 (s, 3H), 0.86 (s, 3H), 0.81 (s, 3H). ¹³C{¹H} (CDCl₃): δ 175.0 (1C), 144.2 (1C), 127.7 (1C), 125.0 (1C), 124.7 (1C), 121.9 (1C), 110.5 (1C), 70.7 (1C), 53.4 (1C), 53.3 (1C), 50.6 (1C), 50.5 (1C), 48.6 (1C), 44.6 (1C), 37.8 (1C), 36.1 (1C), 29.1 (1C), 28.6 (1C), 28.5 (1C), 27.5 (1C), 20.4 (1C), 19.0 (1C), 17.3 (1C). Cald. (C₂₃H₃₂N₂O. 0.1H₂O) %: C, 77.97; H, 9.16; N, 7.91. Found %: C, 77.78; H, 9.09; N, 7.82.

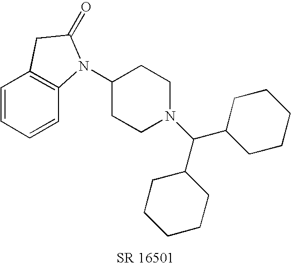

Analytical data for compound SR 16501: HRMS m/e [M+H]: Cald. (C₂₆H₃₈N₂O): 395.3062. Found: 395.3073. ¹H (CDCl₃): δ 7.30-6.92 (m, 4H), 4.21 (tt, J=12.6, 4.1 Hz, 1H), 3.46 (s, 2H), 2.87 (br, 2H), 2.71 (t, J=11.4 Hz, 2H), 2.32 (qd, J=12.0, 4.1 Hz, 2H), 2.01 (t, J=6.0 Hz, 1H), 1.90-0.92 (m, 24H). ¹³C{¹H} (CDCl₃): δ 174.9 (1C), 144.3 (1C), 127.7 (1C), 125.1 (1C), 124.7 (1C), 121.9 (1C), 110.5 (1C), 74.3 (1C), 51.1 (1C), 51.0 (2C), 38.7 (2C), 36.1 (1C), 31.9 (2C), 31.6 (2C), 29.5 (2C), 27.1 (4C), 27.0 (2C).

### EXAMPLE 6

Compound SR 16492 was prepared in a similar manner to the scheme in Example 1, using commercially available tropinone (13) as the starting material:

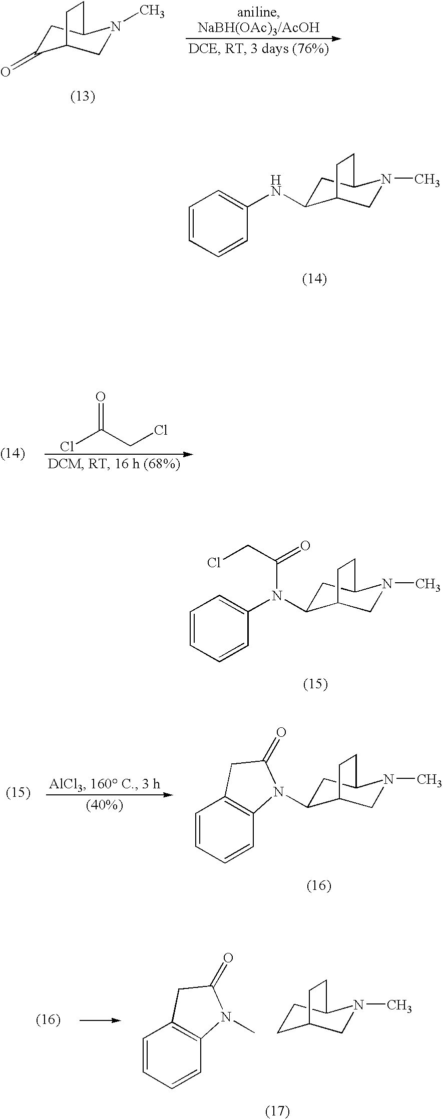

Compound (16) (165 mg, 644 μmol) and proton sponge (28 mg) in DCM (5 mL) at 0° C. was treated with chloroacetyl chloride (105 μL, 644 μmol). The solution was refluxed for 1 hour, cooled and acidified with HCl in ether (1M, 3 drops) and the solvents evaporated. The residue was passed through a 5 g plug of silica gel and eluted with 1:1 EtOAc-hexane (20 mL) and the eluant was evaporated to dryness. The residue was treated with MeOH (5 mL) at reflux for 1 h, and evaporated to dryness. This residue was partitioned between EtOAc and saturated aqueous NaHCO₃. The organic phase was collected, dried (Na₂SO₄) and evaporated to dryness to yield (17), which was used without further purification.

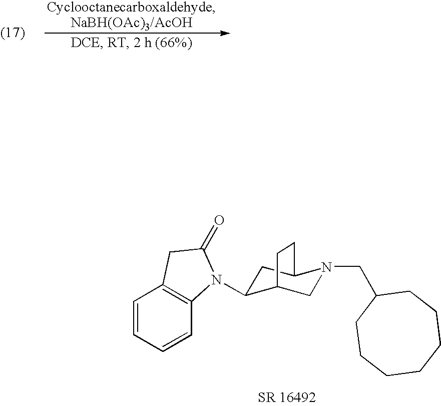

Analytical data for compound SR 16492: ¹H (300 MHz, CDCl₃): δ 7.24-7.19 (m, 2H), 6.99 (d,t, J=0.9, 7.5 Hz, 1H), 6.89 (d, J=7.5 Hz, 1H), 4.69 (m, 1H), 3.45 (s, 2H), 3.36-3.26 (m, 2H), 2.32-2.21 (m, 2H), 2.11-2.04 (m, 2H), 2.00 (d, J=7.5 Hz, 2H), 1.92-1.40 (m, 21 H), 1.30-1.18 (m, 2H). MS (APCI+) m/e 367 (M+H).

### EXAMPLE 7

### ORL1 Activity

The compounds of the invention were evaluated for binding affinity at the ORL1 receptor. Affinity was determined using [³H]N/OFQ binding to membranes derived from CHO cells transfected with human ORL1.

Cell Culture: ORL1-containing CHO cells were produced with cDNA kindly provided by Dr. Brigitte Kieffer of ESBS Universite Louis Pasteur, Strasbourg, France. Each of the cell lines was grown in Dulbecco's Modified Eagle Medium with 10% fetal bovine serum, in the presence of 0.4 mg/ml G418 and 0.1% penicillin/streptomycin, in 100-mm plastic culture dishes.

Receptor Binding: Binding to cell membranes was examined as described in Adapa et al. (1997) Neuropeptides 31:403-408, 1997. Cell membranes were resuspended in 50 mM Tris, pH 7.5, and the suspension incubated with [³H]N/OFQ in a total volume of 1.0 ml, in a 96-well format, for 120 min at 25° C. Samples were filtered over glass fiber filters by using a Wallac cell harvester.

Kᵢ values will be determined by the curve-fitting program Prism, and were calculated from the formula Kᵢ=IC₅₀/(1+L/K_{d}), where K_{d} is the binding affinity of [³H]N/OFQ and L is the concentration of [³H]N/OFQ used. IC₅₀ values were not determined, but can readily be obtained by the curve fitting program Prism.

The resulting data is presented below, where compounds SR 14148, SR 14150, SR 16406, and SR 16407 exhibited particularly favorable Kᵢ values.

TABLE 1

| COMPOUND | Kᵢ (nM) |
|---|---|
| Endogenous N/OFQ | 0.06 |
| SR 16412 | 201 ± 51 |
| SR 14148 | 6.04 ± 0.42 |
| SR 14150 | 1.39 ± 0.42 |
| SR 16405 | 29.3 ± 11.4 |
| SR 16439 | 11.7 ± 1.6 |
| SR 16406 | 4.67 ± 1.96 |
| SR 16407 | 5.64 ± 2.89 |
| SR 16408 | 15.6 ± 1.8 |
| SR 16410 | 253 ± 19 |
| SR 16411 | 135 ± 50 |
| SR 16432 | 22 ± 8.9 |
| SR 16433 | 27.9 ± 8.7 |
| SR 16434 | 7.49 ± 0.78 |
| SR 16476 | 3.96 ± 1.55 |
| SR 16477 | 71.2 ± 20.1 |
| SR 16414 | 15.5 ± 7.1 |
| SR 16501 | >10k |
| SR 16490 | >10k |

### EXAMPLE 8

### Opiate Receptor Activity

The compounds of the invention were evaluated for binding affinity to the μ, κ, and δ opitate receptors, in a manner similar to that in Example 6. The resulting data is presented below:

TABLE 2

| | Ki (nM) | | |
|---|---|---|---|
| COMPOUND | μ | κ | δ |
| Endogenous N/OFQ | 133 | 380 | 267 |
| SR 16412 | 91.1 ± 16 | 84.5 ± 0.8 | ND |
| SR 14148 | 14.4 ± 1.1 | 229 ± 33 | >10K |
| SR 14150 | 29.9 ± 2.1 | 42.7 ± 1.0 | ND |
| SR 16405 | 38.9 ± 9.9 | 55.1 ± 14.9 | ND |
| SR 16439 | 21.2 ± 2.6 | 21.5 ± 2 | ND |
| SR 16406 | 16.4 ± 3.3 | 137 ± 2 | ND |
| SR 16407 | 10.6 ± 1.2 | 52.1 ± 31.5 | ND |
| SR 16408 | 36.7 ± 2.7 | 202 ± 18 | ND |
| SR 16410 | 210 ± 40 | 3869 ± 365 | ND |
| SR 16411 | 20.2 ± 8.2 | 74.4 ± 9.1 | ND |
| SR 16432 | 115 ± 0.9 | 372 ± 0.2 | >10k |
| SR 16433 | 118 ± 1.2 | 318 ± 27 | >10k |
| SR 16434 | 2.70 ± 0.5 | 31.7 ± 4.82 | >10k |
| SR 16476 | 8.0 ± 0.97 | 148 ± 9 | ND |
| SR 16477 | 22.4 ± 6.5 | 369 ± 116 | ND |
| SR 16414 | 27.8 ± 5.4 | 175 ± 3 | ND |
| SR 16501 | 227 ± 80 | 343 ± 92 | ND |

ND = Not determined

### EXAMPLE 9

### GTPγS Activity and Antagonist/Agonist Evaluation

The compounds were evaluated for GTPyS activity. [³⁵S] GTPγS binding was conducted as described in Dooley et al. (1997) J. Pharmacol. Exp. Ther. 283:735-741. Cell membranes were suspended in Buffer A, containing 20 mM HEPES, 10 mM MgCl₂, and 100 mM NaCl, pH 7.4, and sometimes frozen at −70° C. prior to the final centrifugation. For the binding assay, membranes (10-20 mg protein) were incubated with [³⁵S]GTPγS (50 pM), GDP (usually 10 μM), and test compounds, in a total volume of 1 ml, for 60 min at 25° C. Samples were filtered over glass fiber filters and counted as described for the binding assays. Kₑ=[A]/(Dose ratio−1), where [A] is the antagonist concentration. The results are shown in Table 3.

TABLE 3

| | Functional Activity ORL1 [³⁵S]GTPγS | | |
|---|---|---|---|
| COMPOUND | EC₅₀ (nM) | % Stimulation | Kₑ (nM) |
| SR 16412 | 174 ± 51 | 18 ± 2 | |
| SR 14148 | >10k | — | 15.3 ± 1.6 |
| SR 14150 | 19.9 ± 3.4 | 59.1 ± 7.1 | |
| SR 16405 | 92.5 ± 16 | 30.9 ± 3.1 | |
| SR 16439 | ND | | |
| SR 16406 | 74.9 ± 2 | 78 ± 14 | |
| SR 16407 | 16 ± 3 | 62 ± 6 | |
| SR 16408 | 117 ± 43 | 40 ± 8 | |
| SR 16410 | >10k | | |
| SR 16411 | >10k | | |
| SR 16432 | >10k | | ND |
| SR 16433 | >10k | | ND |
| SR 16434 | 28.7 ± 0.6 | 45 ± 5 | |
| SR 16476 | 26.3 ± 8 | 100 | |
| SR 16477 | 117 ± 4 | 65 | |
| SR 16414 | >10k | | 67.1 ± 7.8 |
| SR 16501 | ND | | |

ND = Not determined

A dose response with the full agonist N/OFQ was conducted in each experiment to identify full and partial agonist compounds. The results are shown in Table 4.

TABLE 4

| COMPOUND | LIGAND |
|---|---|
| SR 16412 | agonist |
| SR 14148 | antagonist |
| SR 14150 | agonist |
| SR 16405 | agonist |
| SR 16406 | agonist |
| SR 16407 | agonist |
| SR 16408 | agonist |
| SR 16410 | antagonist |
| SR 16411 | antagonist |
| SR 16432 | antagonist |
| SR 16433 | antagonist |
| SR 16434 | agonist |
| SR 16435, HCl salt | agonist |
| of SR 16434 | |
| SR 16414 | antagonist |

### EXAMPLE 10

### Determination of ORL1-Mediated Inhibition of cAMP Accumulation

The activity of N/OFQ and other ORL 1-active agents is determined by measuring potency for the inhibition of forskolin-stimulated cAMP accumulation in intact CHO cells plated on 24-well plastic plates as described in Dooley et al., supra. To each well is added 0.5 ml buffer with or without an appropriate ORL1 antagonist. Cells are then preincubated, in triplicate, for 10 min at room temperature. After the preincubation, the buffer is aspirated and replaced with fresh buffer containing forskolin (usually 10 μM) and the test compound, with or without antagonist. Cells are then incubated for an additional 10 min. Inhibition induced by 0.1 μM N/OFQ is measured in every experiment as a positive control to determine maximal inhibition. This approach allows for the identification of partial agonists despite the experiment-to-experiment variability in maximal inhibition.

After the incubation, the buffer is aspirated and 0.5 ml of 0.5 M formic acid is added to each well. The formic acid lyses the cells, liberating soluble contents and attaching most of the protein to the plates. The formic acid is left on the plates at least 1 h, then removed and lyophilized. Then 0.5 ml of 0.5 M NaOH is added to each well to solubilize the protein for determination of protein content in each well. The protein is assayed using a BCA Protein Assay kit (Pierce Chemical Co., Rockford, Ill.). The lyophilized residues from each well are suspended in 0.5 ml of 100 mM sodium acetate buffer, pH 4.0, and assayed for cAMP by the protein kinase binding method described in of Gilman (1970) Proc Natl Acad Sci USA 67(1):305-12. Data are expressed as pmol cAMP/mg protein. Each well is assayed individually and the triplicates averaged. IC₅₀ values for inhibition are determined using the program Prism.

### EXAMPLE 11

### Tail Flick Latency Test

Mice are kept on a 12 h light/12 h dark regimen and housed 10 per cage. Tail flick latencies are determined using a Tail Flick Analgesia Instrument (Stoelting). This instrument uses radiant heat, with automatic quantification of tail flick latency, and a 15-s cutoff to prevent damage to the animal's tail. For the assay procedure, response latencies are determined before agonist administration, for baseline values, and again at the time of antinociceptive testing. Failure of the mouse to respond prior to the 15-s cutoff results in assignment of a maximal score. Antinociception will be quantitated by the formula:
% Antinociception=100×(test latency−baseline latency)/(15−baseline latency)

For most studies, compounds are administered via SC or intraperitoneal (IP) injection if appropriate. On some occasions, compounds are delivered by ICV or IT administration. ICV injections are delivered essentially by the method described in Mattia et al. (1991) Pharmacol. Exp. Ther. 258:583-587. Mice are lightly anesthetized with isoflurane and the skin of the scalp cut with a scalpel to reveal the skull. The injections (1-5 μl volume) are placed into the lateral ventricle, 2 mm caudal to the bregma and 1.5 mm lateral to the midline, by using a Hamilton syringe equipped with a 26-gauge needle fitted with a plastic sleeve to prevent more than 2.5 mm penetration beyond the skull surface.

The doses used will depend upon potency of the compounds tested. The experiments will start with low doses (0.01 mg/kg), and behavioral changes as well as an effect on tail flick latency will be observed. Doses will increase until some effects on tail flick latency can be observed, or until other behavioral changes interfere with the analgesia assay. For antagonist assays, N/OFQ is administered by ICV injection at doses of 1, 3, and 10 nmol per mouse. Tail flick latency is initially measured 15, 30, and 60 min after test compound addition. For antagonist experiments, the compound is either administered ICV (or IT) with N/OFQ, or SC, followed 20 min later by an ICV injection of nociceptin, with the determination of tail flick latency after an additional 10 min.

### EXAMPLE 12

### Foot Withdrawal Latency

Effects of ORL1 agonist and antagonist test compounds is examined in rats, in the Bennett CCI model of chronic pain. Rats are purchased from Taconic with sciatic nerves ligated. In these animals, the foot withdrawal latency is measured in one paw, with the contralateral paw as a control. Foot withdrawal latency is measured using a Plantar Analgesia Instrument (Stoelting). The apparatus allows the rat to move freely without restraint, uses radiant heat, allows bilateral testing, and, as with the tail flick apparatus, automatically detects the end point. Animals are placed in the compartments and observed for 2-5 min until they adjust to the change of environment and remain in the resting position. The infrared (IR) source is placed directly beneath the plantar surface of a rear paw of the rat. “Withdrawal latency” is considered to be the time required, to the nearest 0.1 second, for a rat to withdraw the paw from the IR source. The intensity of the IR source is adjusted such that the withdrawal latency for control animals is approximately 10 seconds. In each testing session, three rats are chosen at random from the test groups, and withdrawal latencies measured for each rat in sequence. Eight cycles of measurement are conducted for each set of three animals. The time between cycles will be 45 seconds. A global mean withdrawal latency for similarly treated animals is calculated from the mean withdrawal latencies of the second through seventh measurements of withdrawal latency for each individual animal.

This method is used for test compounds, so that SC injections can be used for drug administration. Control foot withdrawal latencies, as well as a morphine dose response, is determined on each rat prior to the administration of ORL1 agonist and antagonist test compounds.

### EXAMPLE 13

### Place Conditioning (PC) Paradigm

Apparatus: The apparatus consists of rectangular Plexiglas chambers divided into two distinct equal-sized compartments. One compartment has cedar-scented bedding underneath a bar grid floor and all but the front wall is black. The other compartment has pine-scented bedding beneath a mesh floor and all but the front wall is white. The front walls are transparent so that the mouse's behavior can be monitored. During conditioning, the compartments are divided by a solid partition. However, on the PC test day, the solid partition is replaced with a partition that has an opening allowing the animal free access to both compartments. Each compartment is equipped with IR photobeams arranged such that consecutive beam breakages are recorded and used as a measure of locomotor activity.

Testing Procedure: Three 2-day conditioning trials are conducted over 6 consecutive days. On one day of the trial, mice are injected with their respective drug and confined to one of the conditioning compartments for 30 min. On the other day, they are injected with saline and confined to the other compartment for 30 min. The particular compartment paired with the drug and the order of placement into the compartments is counterbalanced.

Locomotor activity is measured daily by an automated photocell system. The incidence of sniffing, rearing, and licking is assessed following the first and last drug injection by recording the presence of these behaviors every 10 seconds throughout the testing period. An observer unaware of the animal's prior drug treatment will record these behaviors.

At 24 h after the last conditioning day, the mice are tested for PC. The solid partition is replaced with a partition containing an opening giving the animal access to both compartments simultaneously for 15 min. The amount of time the animal spends in each compartment is recorded. If the animal spends significantly more time in the drug-paired compartment, this is termed a conditioned place preference and is thought to reflect the rewarding properties of a drug. However, if an animal spends more time in the saline-paired compartment, this is termed a conditioned place aversion and is thought to reflect the aversive properties of a drug.

1. A method for modulating a process mediated by the nociceptin receptor, comprising administering to a mammalian subject an amount of an N-substituted indolin-2-one derivative effective to modulate the process, wherein the indolin-2-one derivative is N-substituted with a nitrogen-containing alicyclic group, the nitrogen atom of said alicyclic group optionally substituted with a cyclic hydrocarbyl substituent.

2. The method of claim 1, wherein the N-substituted indolin-2-one derivative exhibits antagonist activity, as determined by the degree of inhibition of the nociceptin receptor.

3. The method of claim 1, wherein the N-substituted indolin-2-one derivative exhibits agonist activity, as determined by the degree of stimulation of the nociceptin receptor.

4. The method of claim 1, wherein the N-substituted indolin-2-one derivative has a Kᵢ value of less than 50 nanomolar.

5. The method of claim 1, wherein the N-substituted indolin-2-one derivative is selective for the nociceptin receptor.

6. The method of claim 1, wherein the N-substituted indolin-2-one derivative also binds one or more of the μ, κ, and δ opioid receptors.

7. The method of claim 1, wherein the N-substituted indolin-2-one derivative is a piperidyl indolinone.

8. The method of claim 7, wherein the N-substituted indolin-2-one derivative is piperidin-4-yl-1,3-dihydro-indol-2-one.

9. The method of claim 7, wherein the piperidyl ring has one or more substituents selected from hydroxyl, hydroxyalkyl, halo, alkylidene, amino, aminoalkyl, aminocarbonyl, alkylamino, dialkylamino, alkyl, alkenyl, alkynyl, alkoxy, carboxy, carboxyalkyl, alkoxycarbonyl, alkoxycarbonylalkyl, aryl, aralkyl, carbamoyl, carbamoylalkyl, alkylcarbamoyl, alkylcarbamoylalkyl, dialkylcarbamoyl, dialkylcarbamoylalkyl, carbamoylaminoalkyl, alkylcarbamoylaminoalkyl, dialkylcarbamoylaminoalkyl, carbamoyloxyalkyl, alkylcarbamoyloxyalkyl, dialkylcarbamoyloxyalkyl, alkylsulfonylaminoalkyl, aminosulfonylaminoalkyl, alkylaminosulfonylaminoalkyl, dialkylaminosulfonylaminoalkyl, and heteroaryl.

10. The method of claim 7, wherein the indolinone ring has one or more substituents selected from hydroxyl, halo, amino, aminoalkyl, aminocarbonyl, alkylamino, dialkylamino, alkyl, alkenyl, alkoxy, alkyloxycarbonyl, alkylthio, aryloxy, aryl, aralkyl, arylthio, carboxyl, cycloalkyl, cycloalkylalkyl, heteroaryl, and heteroarylalkyl.

11. The method of claim 1, wherein the process is the in vivo modulation of a drug or alcohol abuse disorder, the in vivo modulation of pain, the in vivo modulation of anxiety and stress disorders, the in vivo modulation of eating disorders, the in vivo modulation of schizophrenia, the in vivo modulation of Parkinsonism, the in vivo modulation of depression, the in vivo modulation of cognitive dysfunction, or the in vivo modulation of cognitive brain function.

12. The method of claim 1 1, wherein the process is the in vivo modulation of a drug or alcohol abuse disorder, the in vivo modulation of pain, or the in vivo modulation of anxiety.

13. A method for modulating the nociceptin receptor in a patient, comprising administering to the patient a therapeutically effective amount of a compound of formula (I): 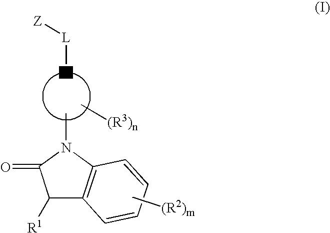
wherein:
m is an integer in the range of zero to 3 inclusive;
n is an integer in the range of zero to 2 inclusive; 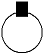
is a nitrogen-containing heterocyclic group having 3-14 carbon atoms;
R¹ is selected from hydrogen, hydroxyl, halo, haloalkyl, amino, aminoalkyl, aminocarbonyl, alkylamino, dialkylamino, alkyl, alkenyl, alkoxy, alkoxycarbonyl, aryl, and aralkyl, or can be taken together with R² to form a cyclic group;
R² is selected from hydroxyl, halo, haloalkyl, amino, aminoalkyl, aminocarbonyl, alkylamino, dialkylamino, alkyl, alkenyl, alkoxy, alkoxycarbonyl, alkylthio, aryloxy, aryl, aralkyl, arylthio, carboxy, cycloalkyl, cycloalkylalkyl, heteroaryl, and heteroarylalkyl, or two R² substituents taken together can form a cyclic structure, and further wherein when m is greater than 1, the R² groups may be the same or different;
R³ is selected from hydrogen, hydroxyl, hydroxyalkyl, halo, haloalkyl, alkylidene, amino, aminoalkyl, aminocarbonyl, alkylamino, dialkylamino, alkyl, alkenyl, alkynyl, alkoxy, carboxy, carboxyalkyl, alkoxycarbonyl, alkoxycarbonylalkyl, carbamoyl, carbamoylalkyl, alkylcarbamoyl, alkylcarbamoylalkyl, dialkylcarbamoyl, dialkylcarbamoylalkyl, carbamoylaminoalkyl, alkylcarbamoylaminoalkyl, dialkylcarbamoylaminoalkyl, carbamoyloxyalkyl, alkylcarbamoyloxyalkyl, dialkylcarbamoyloxyalkyl, alkylsulfonylaminoalkyl, aminosulfonylaminoalkyl, alkylaminosulfonylaminoalkyl, dialkylaminosulfonylaminoalkyl, and heteroaryl, and wherein when n is 2, the two R³ groups may be the same or different;
L is —(CHR⁴)ₚ—, —CH₂—Xᵥ—, or —C(═NH)—NR⁵—;
p is an integer in the range of zero to 4 inclusive;
v is an integer in the range of 1 to 3 inclusive;
R⁴ is selected from hydrogen, hydroxyl, halo, amino, aminoalkyl, alkylamino, dialkylamino, alkyl, cycloalkyl, cycloalkylalkyl, alkenyl, alkoxy, aryl and aralkyl, wherein when p is greater than 1, the R⁴ may be the same or different;
R⁵ is selected from hydrogen, alkyl, cycloalkyl, cycloalkylalkyl, alkenyl, alkoxy, aryl and aralkyl;
X is independently CH₂, NR⁶ or O, wherein at least one X, if v is greater than 1, is NR⁶ or O;
R⁶ is selected from hydrogen, alkyl, cycloalkyl, cycloalkylalkyl, alkenyl, alkoxy, aryl and aralkyl, and. In some preferred embodiments, when L is —(CHR⁴)ₚ—, p is 0 and so the R⁴ group; and
Z is selected from hydrogen and cyclic hydrocarbyl, optionally substituted with one or more substituents, with the proviso that when p is zero, , or a mono-, bi- or tri-cyclic hydrocarbyl, optionally substituted with one or more substituents, with the proviso that when p is zero, Z is other than hydrogen.

14. The method of claim 13, wherein the nitrogen-containing heterocyclic group is monocyclic.

15. The method of claim 14, wherein the nitrogen-containing heterocyclic group is piperidyl.

16. The method of claim 13, wherein nitrogen-containing heterocyclic group is bicyclic.

17. The method of claim 16, wherein the nitrogen-containing heterocyclic group is selected from:

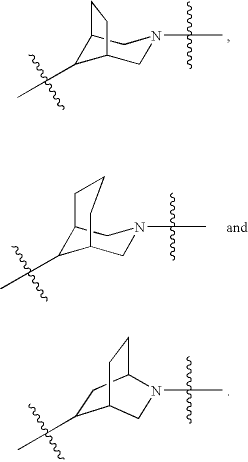

18. The method of claim 13, wherein R¹ is selected from hydrogen and alkyl.

19. The method of claim 13, wherein m is 0.

20. The method of claim 13, wherein m is 1, 2, or 3.

21. The method of claim 20, wherein R² is selected from alkyl and alkoxycarbonyl.

22. The method of claim 13, wherein n is 0.

23. The method of claim 13, wherein n is 1 or 2.

24. The method of claim 23, wherein R³ is selected from alkyl and alkoxycarbonyl.

25. The method of claim 13, wherein L is —(CHR⁴)ₚ—, and p is 0.

26. The method of claim 13, wherein L is —(CHR⁴)ₚ—, and p is 1, 2, or 3.

27. The method of claim 26, wherein R⁴ is selected from hydrogen, alkyl, and cycloalkyl.

28. The method of claim 13, wherein Z is substituted with one or more substituents selected from hydroxyl, halo, haloalkyl, amino, aminoalkyl, alkylamino, dialkylamino, alkyl, alkenyl, alkoxy, aryl and aralkyl.

29. The method of claim 13, wherein Z is monocyclic.

30. The method of claim 29, wherein Z has the structure 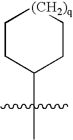
where q is an integer from 0-5.

31. The method of claim 30, wherein: the nitrogen-containing heterocyclic group is piperidyl; R¹ is H; m and n are 0; L is —(CHR⁴)ₚ—; p is 1; R⁴ is H; and q is 3.

32. The method of claim 30, wherein: the nitrogen-containing heterocyclic group is piperidyl; R¹ is H; m and n are 0; L is —(CHR⁴)ₚ—; p is 0; and q is 3.

33. The method of claim 30, wherein: the nitrogen-containing heterocyclic group is piperidyl; R¹ is H; m and n are 0; L is —(CHR⁴)ₚ—; p is 0; q is 1; and Z is substituted with a C₁₋₆ alkyl group.

34. The method of claim 29, wherein the C₁₋₆ alkyl group is phenyl.

35. The method of claim 34, wherein the nitrogen-containing heterocyclic group is piperidyl; R¹ is H; m and n are 0; L is —(CHR⁴)ₚ—; p is 1; and R⁴ is H.

36. The method of claim 34, wherein Z is substituted with a cycloalkyl group and has the structure: 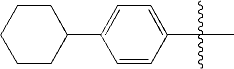
and wherein the nitrogen-containing heterocyclic group is piperidyl, R¹ is H, m and n are 0, L is —(CHR⁴)ₚ—, p is 1, and R⁴ is —C₁₋₆ alkyl.

37. The method of claim 13, wherein Z is a bicyclic hydrocarbyl substituent.

38. The method of claim 37, wherein the bicyclic hydrocarbyl substituent is a fused ring system.

39. The method of claim 38, wherein the fused ring system is selected from: 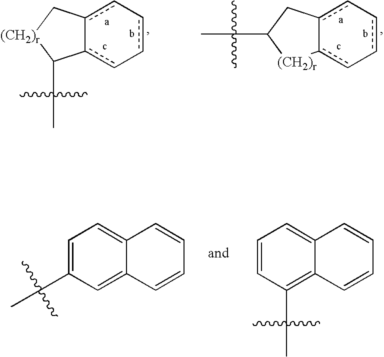
where r is an integer from 1-2; and a, b, and c, are optional double bonds.

40. The method of claim 39, wherein the aliphatic nitrogen-containing group is piperidyl, R¹ is H; m and n are 0; L is —(CHR⁴)ₚ—; p is 0; r is 2; a, b, and c, are double bonds; and the fused ring system is:

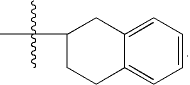

41. The method of claim 39, wherein the aliphatic nitrogen-containing group is piperidyl, R¹ is H; m and n are 0; L is —(CHR⁴)ₚ—; p is 0; r is 2; a, b, and c, are double bonds; and the fused ring system is:

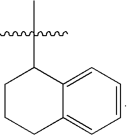

42. The method of claim 39, wherein the aliphatic nitrogen-containing group is piperidyl, R¹ is H; m and n are 0; L is —(CHR⁴)ₚ—; p is 0; r is 2; a, b, and c, are absent; and the fused ring system is:

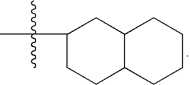

43. The method of claim 39, wherein the aliphatic nitrogen-containing group is piperidyl, R¹ is H; m and n are 0; L is —(CHR⁴)ₚ—; p is 0; r is 2; a, b, and c, are absent; and the fused ring system is:

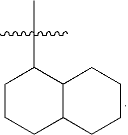

44. The method of claim 38, wherein the fused ring system is selected from: 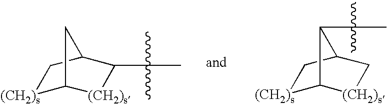
where s and s′ are integers from 0-2.

45. The method of claim 44, wherein the aliphatic nitrogen-containing group is piperidyl, R¹ is H; m and n are 0; L is —(CHR⁴)ₚ—; p is 1; R⁴ is H; s and s′ are 1; and the fused ring system is:

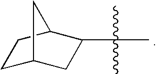

46. The method of claim 44, wherein the aliphatic nitrogen-containing group is piperidyl, R¹ is H; m and n are 0; L is —(CHR⁴)ₚ—; p is 0; s and s′ are 2; and the bi-cyclic hydrocarbyl is:

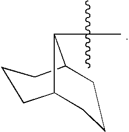

47. The method of claim 44, wherein the aliphatic nitrogen-containing group is piperidyl, R¹ is H; m and n are 0; L is —(CHR⁴)ₚ—; p is 1; R⁴ is H; s is 0; s′ is 2; and the fused ring system is:

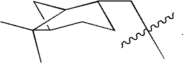

48. A compound having the structure of formula (Ia): 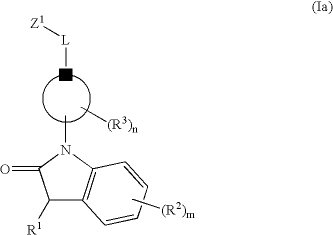
wherein:
m is an integer in the range of zero to 3 inclusive;
n is an integer in the range of zero to 2 inclusive; 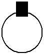
is a nitrogen-containing heterocyclic group having 3-14 carbon atoms;
R¹ is selected from hydrogen, hydroxyl, halo, haloalkyl, amino, aminoalkyl, aminocarbonyl, alkylamino, dialkylamino, alkyl, alkenyl, alkoxy, alkoxycarbonyl, aryl, and aralkyl, or can be taken together with R² to form a cyclic group;
R² is selected from hydroxyl, halo, haloalkyl, amino, aminoalkyl, aminocarbonyl, alkylamino, dialkylamino, alkyl, alkenyl, alkoxy, alkoxycarbonyl, alkylthio, aryloxy, aryl, aralkyl, arylthio, carboxy, cycloalkyl, cycloalkylalkyl, heteroaryl, and heteroarylalkyl, or two R² substituents taken together can form a cyclic structure, and further wherein when m is greater than 1, the R² groups may be the same or different;
R³ is selected from hydrogen, hydroxyl, hydroxyalkyl, halo, haloalkyl, alkylidene, amino, aminoalkyl, aminocarbonyl, alkylamino, dialkylamino, alkyl, alkenyl, alkynyl, alkoxy, carboxy, carboxyalkyl, alkoxycarbonyl, alkoxycarbonylalkyl, carbamoyl, carbamoylalkyl, alkylcarbamoyl, alkylcarbamoylalkyl, dialkylcarbamoyl, dialkylcarbamoylalkyl, carbamoylaminoalkyl, alkylcarbamoylaminoalkyl, dialkylcarbamoylaminoalkyl, carbamoyloxyalkyl, alkylcarbamoyloxyalkyl, dialkylcarbamoyloxyalkyl, alkylsulfonylaminoalkyl, aminosulfonylaminoalkyl, alkylaminosulfonylaminoalkyl, dialkylaminosulfonylaminoalkyl, and heteroaryl, and wherein when n is 2, the two R³ groups may be the same or different;
L is —(CHR⁴)ₚ—, —CH₂—Xᵥ—, or —C(═NH)—NR⁵—;
p is an integer in the range of zero to 4 inclusive;
v is an integer in the range of 1 to 3 inclusive;
R⁴ is selected from hydrogen, hydroxyl, halo, amino, aminoalkyl, alkylamino, dialkylamino, alkyl, cycloalkyl, cycloalkylalkyl, alkenyl, alkoxy, aryl and aralkyl, wherein when p is greater than 1, the R⁴ may be the same or different;
R⁵is selected from hydrogen, alkyl, cycloalkyl, cycloalkylalkyl, alkenyl, alkoxy, aryl and aralkyl
X is independently CH₂, NR⁶ or O, wherein at least one X, if v is greater than 1, is NR⁶ or O;
R⁶ is selected from hydrogen, alkyl, cycloalkyl, cycloalkylalkyl, alkenyl, alkoxy, aryl and aralkyl, and. In some preferred embodiments, when L is —(CHR⁴)ₚ—, p is 0 and so the R⁴ group; and
Z¹ is monocyclic hydrocarbyl,
with the proviso that when 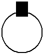
is piperidyl, Z¹ is cyclohexyl, L is —(CHR⁴)ₚ—, and p is 1, then R⁴ is other than hydrogen.

49. The compound of claim 48, wherein Z¹ is substituted with one or more substituents selected from hydroxyl, halo, haloalkyl, amino, aminoalkyl, alkylamino, dialkylamino, alkyl, alkenyl, alkoxy, aryl, and aralkyl.

50. The compound of claim 48, wherein Z¹ has the structure 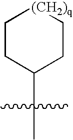
where q is an integer from 0-5.

51. The compound of claim 50, wherein the nitrogen-containing heterocyclic group is piperidyl; R¹ is H; m and n are 0; L is —(CHR⁴)ₚ—; p is 1; R⁴ is H; and q is 3.

52. The compound of claim 50, wherein the nitrogen-containing heterocyclic group is piperidyl; R¹ is H; m and n are 0; L is —(CHR⁴)ₚ—; p is 0; and q is 3.

53. The compound of claim 50, wherein the nitrogen-containing heterocyclic group is piperidyl; R¹ is H; m and n are 0; L is —(CHR⁴)ₚ—; p is 0; q is 1; and Z has a —C₁₋₆ alkyl at the 4-position.

54. The compound of claim 50, wherein the nitrogen-containing heterocyclic group is piperidyl; R¹ is H; m and n are 0; L is —(CHR⁴)ₚ—; p is 1; R⁴ is cyclohexyl; and q is 1.

55. A pharmaceutical composition comprising a therapeutically effective amount of the compound of claim 48 in combination with a pharmaceutically acceptable carrier.

56. A compound having the structure of formula (Ib): 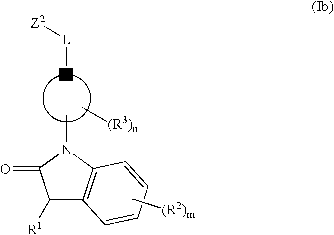
wherein:
m is an integer in the range of zero to 3 inclusive;
n is an integer in the range of zero to 2 inclusive; 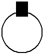
is a nitrogen-containing heterocyclic group having 3-14 carbon atoms;
R¹ is selected from hydrogen, hydroxyl, halo, haloalkyl, amino, aminoalkyl, aminocarbonyl, alkylamino, dialkylamino, alkyl, alkenyl, alkoxy, alkoxycarbonyl, aryl, and aralkyl, or can be taken together with R² to form a cyclic group;
R² is selected from hydroxyl, halo, haloalkyl, amino, aminoalkyl, aminocarbonyl, alkylamino, dialkylamino, alkyl, alkenyl, alkoxy, alkoxycarbonyl, alkylthio, aryloxy, aryl, aralkyl, arylthio, carboxy, cycloalkyl, cycloalkylalkyl, heteroaryl, and heteroarylalkyl, or two R² substituents taken together can form a cyclic structure, and further wherein when m is greater than 1, the R² groups may be the same or different;
R³ is selected from hydrogen, hydroxyl, hydroxyalkyl, halo, haloalkyl, alkylidene, amino, aminoalkyl, aminocarbonyl, alkylamino, dialkylamino, alkyl, alkenyl, alkynyl, alkoxy, carboxy, carboxyalkyl, alkoxycarbonyl, alkoxycarbonylalkyl, carbamoyl, carbamoylalkyl, alkylcarbamoyl, alkylcarbamoylalkyl, dialkylcarbamoyl, dialkylcarbamoylalkyl, carbamoylaminoalkyl, alkylcarbamoylaminoalkyl, dialkylcarbamoylaminoalkyl, carbamoyloxyalkyl, alkylcarbamoyloxyalkyl, dialkylcarbamoyloxyalkyl, alkylsulfonylaminoalkyl, aminosulfonylaminoalkyl, alkylaminosulfonylaminoalkyl, dialkylaminosulfonylaminoalkyl, and heteroaryl, and wherein when n is 2, the two R³ groups may be the same or different;
L is —(CHR⁴)ₚ—, —C—Xᵥ—, or —C(NH)—NR⁵—; where p is an integer from 0-4, and R⁴ is independently selected from hydrogen, hydroxyl, halo, amino, aminoalkyl, alkylamino, dialkylamino, alkyl, cycloalkyl, cycloalkylalkyl, alkenyl, alkoxy, aryl and aralkyl; v is an integer from 1-3, and X is independently C, NR⁶ or 0, wherein at least one X is NR⁶ or 0, and R⁶ is selected from hydrogen, alkyl, cycloalkyl, cycloalkylalkyl, alkenyl, alkoxy, aryl and aralkyl; and R⁵ is selected from hydrogen, alkyl, cycloalkyl, cycloalkylalkyl, alkenyl, alkoxy, aryl and aralkyl; and
Z² is a mono-cyclic unsaturated hydrocarbyl, optionally substituted with one or more substituents;
with the proviso that when 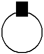
is piperidyl, Z² is phenyl, L is —(CHR⁴)ₚ—, and p is 1, then R⁴ is not hydrogen.

57. The compound of claim 56, wherein Z² is substituted with one or more substituents selected from hydroxyl, halo, haloalkyl, amino, aminoalkyl, alkylamino, dialkylamino, alkyl, alkenyl, alkoxy, aryl and aralkyl.

58. The compound of claim 56, wherein Z² is:

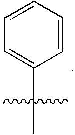

59. The compound of claim 58, wherein Z² is: 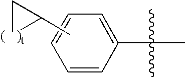
where t is an integer from 1-6.

60. The compound of claim 59, wherein the aliphatic nitrogen-containing heterocyclic group is piperidyl, R¹ is H; m and n are 0; L is —(CHR⁴)ₚ—; p is 1; t is 4; and R⁴ is —C₁₋₆ alkyl.

61. A pharmaceutical composition comprising a therapeutically effective amount of the compound of claim 56 in combination with a pharmaceutically acceptable carrier.

62. A compound having the structure of formula (Ic): 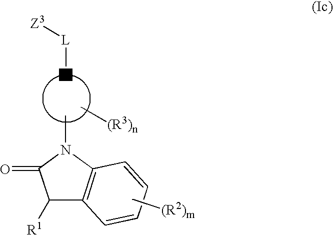
wherein:
m is an integer in the range of zero to 3 inclusive;
n is an integer in the range of zero to 2 inclusive; 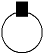
is a nitrogen-containing heterocyclic group having 3-14 carbon atoms;
R¹ is selected from hydrogen, hydroxyl, halo, haloalkyl, amino, aminoalkyl, aminocarbonyl, alkylamino, dialkylamino, alkyl, alkenyl, alkoxy, alkoxycarbonyl, aryl, and aralkyl, or can be taken together with R² to form a cyclic group;
R² is selected from hydroxyl, halo, haloalkyl, amino, aminoalkyl, aminocarbonyl, alkylamino, dialkylamino, alkyl, alkenyl, alkoxy, alkoxycarbonyl, alkylthio, aryloxy, aryl, aralkyl, arylthio, carboxy, cycloalkyl, cycloalkylalkyl, heteroaryl, and heteroarylalkyl, or two R² substituents taken together can form a cyclic structure, and further wherein when m is greater than 1, the R² groups may be the same or different;
R³ is selected from hydrogen, hydroxyl, hydroxyalkyl, halo, haloalkyl, alkylidene, amino, aminoalkyl, aminocarbonyl, alkylamino, dialkylamino, alkyl, alkenyl, alkynyl, alkoxy, carboxy, carboxyalkyl, alkoxycarbonyl, alkoxycarbonylalkyl, carbamoyl, carbamoylalkyl, alkylcarbamoyl, alkylcarbamoylalkyl, dialkylcarbamoyl, dialkylcarbamoylalkyl, carbamoylaminoalkyl, alkylcarbamoylaminoalkyl, dialkylcarbamoylaminoalkyl, carbamoyloxyalkyl, alkylcarbamoyloxyalkyl, dialkylcarbamoyloxyalkyl, alkylsulfonylaminoalkyl, aminosulfonylaminoalkyl, alkylaminosulfonylaminoalkyl, dialkylaminosulfonylaminoalkyl, and heteroaryl, and wherein when n is 2, the two R³ groups may be the same or different;
L is —(CHR⁴)ₚ—, —C—Xᵥ—, or —C(NH)—NR⁵—; where p is an integer from 0-4, and R⁴ is independently selected from hydrogen, hydroxyl, halo, amino, aminoalkyl, alkylamino, dialkylamino, alkyl, cycloalkyl, cycloalkylalkyl, alkenyl, alkoxy, aryl and aralkyl; v is an integer from 1-3, and X is independently C, NR⁶ or 0, wherein at least one X is NR ⁶or 0, and R⁶ is selected from hydrogen, alkyl, cycloalkyl, cycloalkylalkyl, alkenyl, alkoxy, aryl and aralkyl; and R⁵ is selected from hydrogen, alkyl, cycloalkyl, cycloalkylalkyl, alkenyl, alkoxy, aryl and aralkyl; and
Z³ is a bi-cyclic hydrocarbyl, optionally substituted with one or more substituents;
with the proviso that when 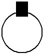
is piperidyl, Z³ is tetrahydronaphthyl or napthyl, L is —(CHR⁴)ₚ—, and p is 1, then R⁴ is not hydrogen.

63. The compound of claim 62, wherein Z³ is substituted with one or more substituents selected from hydroxyl, halo, haloalkyl, amino, aminoalkyl, alkylamino, dialkylamino, alkyl, alkenyl, alkoxy, aryl and aralkyl.

64. The compound of claim 62, wherein Z³ is a fused ring system.

65. The compound of claim 64, wherein the fused ring system is selected from: 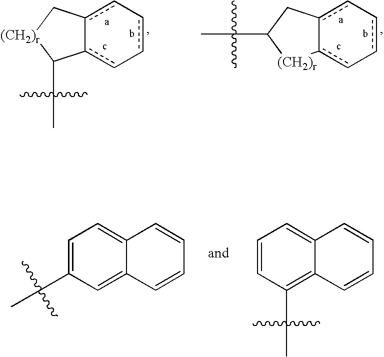
where r is an integer from 1-2; and a, b, and c, are optional double bonds.

66. The compound of claim 65, wherein the aliphatic nitrogen-containing group is piperidyl, R¹ is H; m and n are 0; L is —(CHR⁴)ₚ—; p is 0; r is 2; a, b, and c, are double bonds; and the fused ring system is:

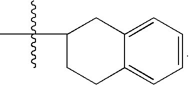

67. The compound of claim 65, wherein the aliphatic nitrogen-containing heterocyclic group is piperidyl, R¹ is H; m and n are 0; L is —(CHR⁴)ₚ—; p is 0; r is 2; a, b, and c, are double bonds; and the fused ring system is:

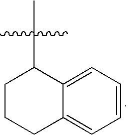

68. The compound of claim 65, wherein the aliphatic nitrogen-containing group is piperidyl, R¹ is H; m and n are 0; L is —(CHR⁴)ₚ—; p is 0; r is 2, a, b, and c, are absent; and the fused ring system is:

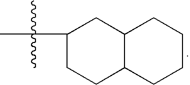

69. The compound of claim 65, wherein the aliphatic nitrogen-containing group is piperidyl, R¹ is H; m and n are 0; L is —(CHR⁴)ₚ—; p is 0, r is 2, a, b, and c, are absent; and the fused ring system is:

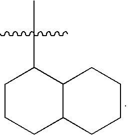

70. The compound of claim 65, wherein the fused ring system is selected from: 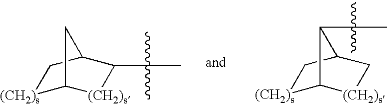
where s and s′ are integers from 0-2.

71. The compound of claim 70, wherein the aliphatic nitrogen-containing group is piperidyl, R¹ is H; m and n are 0; L is —(CHR⁴)ₚ—; p is 1; R⁴ is H; s and s′ are 1; and the fused ring system is:

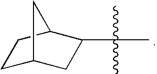

72. The compound of claim 70, wherein the aliphatic nitrogen-containing group is piperidyl, R¹ is H; m and n are 0; L is —(CHR⁴)ₚ—; p is 0; s and s′ are 1; and the fused ring system is:

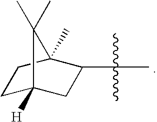

73. The compound of claim 70, wherein the aliphatic nitrogen-containing group is piperidyl, R¹ is H; m and n are 0; L is —(CHR⁴)ₚ—; p is 0; s and s′ are 2; and the bi-cyclic hydrocarbyl is:

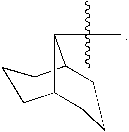

74. The compound of claim 70, wherein the aliphatic nitrogen-containing group is piperidyl, R¹ is H; m and n are 0; L is —(CHR⁴)ₚ—; p is 1; R⁴ is H; s is 0; s′ is 2; and the fused ring system is:

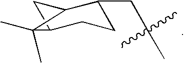

75. A pharmaceutical composition comprising a therapeutically effective amount of the compound of claim 62 in combination with a pharmaceutically acceptable carrier.

76. A compound having the structure of formula (Id): 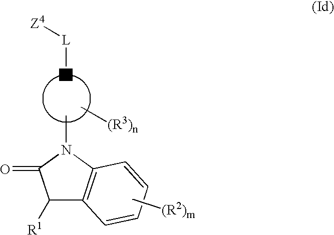
wherein:
m is an integer in the range of zero to 3 inclusive;
n is an integer in the range of zero to 2 inclusive; 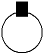
is a nitrogen-containing heterocyclic group having 3-14 carbon atoms;
R¹ is selected from hydrogen, hydroxyl, halo, haloalkyl, amino, aminoalkyl, aminocarbonyl, alkylamino, dialkylamino, alkyl, alkenyl, alkoxy, alkoxycarbonyl, aryl, and aralkyl, or can be taken together with R² to form a cyclic group;
R² is selected from hydroxyl, halo, haloalkyl, amino, aminoalkyl, aminocarbonyl, alkylamino, dialkylamino, alkyl, alkenyl, alkoxy, alkoxycarbonyl, alkylthio, aryloxy, aryl, aralkyl, arylthio, carboxy, cycloalkyl, cycloalkylalkyl, heteroaryl, and heteroarylalkyl, or two R² substituents taken together can form a cyclic structure, and further wherein when m is greater than 1, the R² groups may be the same or different;
R³ is selected from hydrogen, hydroxyl, hydroxyalkyl, halo, haloalkyl, alkylidene, amino, aminoalkyl, aminocarbonyl, alkylamino, dialkylamino, alkyl, alkenyl, alkynyl, alkoxy, carboxy, carboxyalkyl, alkoxycarbonyl, alkoxycarbonylalkyl, carbamoyl, carbamoylalkyl, alkylcarbamoyl, alkylcarbamoylalkyl, dialkylcarbamoyl, dialkylcarbamoylalkyl, carbamoylaminoalkyl, alkylcarbamoylaminoalkyl, dialkylcarbamoylaminoalkyl, carbamoyloxyalkyl, alkylcarbamoyloxyalkyl, dialkylcarbamoyloxyalkyl, alkylsulfonylaminoalkyl, aminosulfonylaminoalkyl, alkylaminosulfonylaminoalkyl, dialkylaminosulfonylaminoalkyl, and heteroaryl, and wherein when n is 2, the two R³ groups may be the same or different;
L is —(CHR⁴)ₚ—, —C—Xᵥ—, or —C(NH)—NR⁵—; where p is an integer from 0-4, and R⁴ is independently selected from hydrogen, hydroxyl, halo, amino, aminoalkyl, alkylamino, dialkylamino, alkyl, cycloalkyl, cycloalkylalkyl, alkenyl, alkoxy, aryl and aralkyl; v is an integer from 1-3, and X is independently C, NR⁶ or 0, wherein at least one X is NR⁶ or O, and R⁶ is selected from hydrogen, alkyl, cycloalkyl, cycloalkylalkyl, alkenyl, alkoxy, aryl and aralkyl; and R⁵ is selected from hydrogen, alkyl, cycloalkyl, cycloalkylalkyl, alkenyl, alkoxy, aryl and aralkyl; and
Z⁴ is a tri-cyclic hydrocarbyl, optionally substituted with one or more substituents.

77. The compound of claim 76, wherein Z⁴ is substituted with one or more substituents selected from hydroxyl, halo, haloalkyl, amino, aminoalkyl, alkylamino, dialkylamino, alkyl, alkenyl, alkoxy, aryl and aralkyl.

78. The compound of claim 76, wherein Z⁴ is:

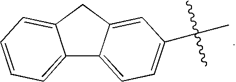

79. The compound of claim 78, wherein the aliphatic nitrogen-containing heterocyclic group is piperidyl, R¹ is H; m and n are 0; L is —(CHR⁴)ₚ—; p is 1; and R⁴ is —C₁₋₆ alkyl.

80. A pharmaceutical composition comprising a therapeutically effective amount of the compound of claim 76 in combination with a pharmaceutically acceptable carrier.

81. A compound having the structure of formula (le): 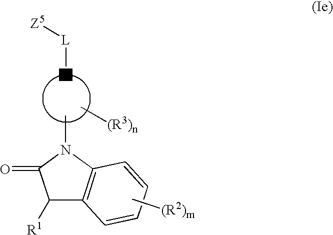
wherein:
m is an integer in the range of zero to 3 inclusive;
n is an integer in the range of zero to 2 inclusive; 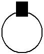
is a nitrogen-containing heterocyclic group having 3-14 carbon atoms;
R¹ is selected from hydrogen, hydroxyl, halo, haloalkyl, amino, aminoalkyl, aminocarbonyl, alkylamino, dialkylamino, alkyl, alkenyl, alkoxy, alkoxycarbonyl, aryl, and aralkyl, or can be taken together with R² to form a cyclic group;
R² is selected from hydroxyl, halo, haloalkyl, amino, aminoalkyl, aminocarbonyl, alkylamino, dialkylamino, alkyl, alkenyl, alkoxy, alkoxycarbonyl, alkylthio, aryloxy, aryl, aralkyl, arylthio, carboxy, cycloalkyl, cycloalkylalkyl, heteroaryl, and heteroarylalkyl, or two R² substituents taken together can form a cyclic structure, and further wherein when m is greater than 1, the R² groups may be the same or different;
R³ is selected from hydrogen, hydroxyl, hydroxyalkyl, halo, haloalkyl, alkylidene, amino, aminoalkyl, aminocarbonyl, alkylamino, dialkylamino, alkyl, alkenyl, alkynyl, alkoxy, carboxy, carboxyalkyl, alkoxycarbonyl, alkoxycarbonylalkyl, carbamoyl, carbamoylalkyl, alkylcarbamoyl, alkylcarbamoylalkyl, dialkylcarbamoyl, dialkylcarbamoylalkyl, carbamoylaminoalkyl, alkylcarbamoylaminoalkyl, dialkylcarbamoylaminoalkyl, carbamoyloxyalkyl, alkylcarbamoyloxyalkyl, dialkylcarbamoyloxyalkyl, alkylsulfonylaminoalkyl, aminosulfonylaminoalkyl, alkylaminosulfonylaminoalkyl, dialkylaminosulfonylaminoalkyl, and heteroaryl, and wherein when n is 2, the two R³ groups may be the same or different;
L is —(CHR⁴)ₚ—, —C—Xᵥ—, or —C(NH)—NR⁵—; where p is an integer from 0-4, and R⁴ is independently selected from hydrogen, hydroxyl, halo, amino, aminoalkyl, alkylamino, dialkylamino, alkyl, cycloalkyl, cycloalkylalkyl, alkenyl, alkoxy, aryl and aralkyl; v is an integer from 1-3, and X is independently C, NR⁶ or O, wherein at least one X is NR⁶ or O, and R⁶ is selected from hydrogen, alkyl, cycloalkyl, cycloalkylalkyl, alkenyl, alkoxy, aryl and aralkyl; and R⁵ is selected from hydrogen, alkyl, cycloalkyl, cycloalkylalkyl, alkenyl, alkoxy, aryl and aralkyl; and
Z⁵ is hydrogen.

82. A pharmaceutical composition comprising a therapeutically effective amount of the compound of claim 81 in combination with a pharmaceutically acceptable carrier.

83. A method for treating an individual with a drug or alcohol abuse disorder, comprising administering to the individual a therapeutically effective amount of an N-substituted indolin-2-one derivative, wherein the indolin-2-one derivative is N-substituted with a nitrogen-containing alicyclic group, the nitrogen atom of said alicyclic group optionally substituted with a cyclic hydrocarbyl substituent.]

84. A method for treating an individual suffering from pain, comprising administering to the individual a therapeutically effective amount of an N-substituted indolin-2-one derivative, wherein the indolin-2-one derivative is N-substituted with a nitrogen-containing alicyclic group, the nitrogen atom of said alicyclic group optionally substituted with a cyclic hydrocarbyl substituent.

85. A method for preventing or treating anxiety and stress disorders in an individual, comprising administering to the individual a therapeutically effective amount of an N-substituted indolin-2-one derivative, wherein the indolin-2-one derivative is N-substituted with a nitrogen-containing alicyclic group, the nitrogen atom of said alicyclic group optionally substituted with a cyclic hydrocarbyl substituent.

86. A method for treating or preventing eating disorders, schizophrenia, Parkinsonism, depression, cognitive dysfunction, or the amelioration of reduced cognitive brain function, comprising administering to an individual in need thereof, a therapeutically effective amount of an N-substituted indolin-2-one derivative, wherein the indolin-2-one derivative is N-substituted with a nitrogen-containing alicyclic group, the nitrogen atom of said alicyclic group optionally substituted with a cyclic hydrocarbyl substituent.

87. A method for treating or preventing hyponatremia, hypokalemia, a water retaining condition, multiple organ failure, hypertension, or edema, comprising administering to an individual in need thereof, a therapeutically effective amount of an N-substituted indolin-2-one derivative, wherein the indolin-2-one derivative is N-substituted with a nitrogen-containing alicyclic group, the nitrogen atom of said alicyclic group optionally substituted with a cyclic hydrocarbyl substituent.
